(19) United States
(12) Patent Application Publication
(10) Pub. No.: US 2022/0365083 A1
(43) Pub. Date: Nov. 17, 2022

(54) METHOD FOR DETECTING LUPUS NEPHRITIS

(71) Applicant: Rheinisch-Westfälische Technische Hochschule (RWTH) Aachen, Aachen (DE)

(72) Inventors: Ute RAFFETSEDER, Aachen (DE); Daniel BREITKOPF, Aachen (DE); Thomas RAUEN, Aachen (DE); Vera JANKOWSKI, Roetgen (DE); Jürgen FLOEGE, Aachen (DE); Tammo OSTENDORF, Aachen (DE)

(73) Assignee: Rheinisch-Westfälische Technische Hochschule (RWTH) Aachen, Aachen (DE)

(21) Appl. No.: 17/767,014

(22) PCT Filed: Oct. 7, 2020
(86) PCT No.: PCT/EP2020/078033

§ 371 Date: Apr. 6, 2022

(30) Foreign Application Priority Data
Oct. 10, 2019 (EP) 19202446.1

(51) Int. Cl. G01N 33/564

(52) U.S. Cl. CPC G01N 33/564

(57) ABSTRACT

The present invention relates to a method for diagnosing lupus nephritis in an individual suffering from systemic lupus erythematosus (SLE), said method conducted in vitro comprising identifying a protein that comprises an YB-1-like cold-shock domain which bears one or more guanidinylated lysinyl moieties (YB-1-G) in a sample S obtained from the individual. Furthermore, the present invention also refers to treating or preventing lupus nephritis in an individual, wherein lupus nephritis has been diagnosed in the individual by means of a method of the present invention.

The present invention relates to a method for diagnosing lupus nephritis in an individual suffering from systemic lupus erythematosus (SLE), said method conducted in vitro comprising identifying a protein that comprises an YB-1-like cold-shock domain which bears one or more guanidinylated lysinyl moieties (YB-1-G) in a sample S obtained from the individual. Furthermore, the present invention also refers to treating or preventing lupus nephritis in an individual, wherein lupus nephritis has been diagnosed in the individual by means of a method of the present invention.

Lupus nephritis (LN) is a severe pathological condition, which is associated with a significantly increased morbidity and increased mortality in lupus patients. Hundred thousands of individuals worldwide suffer from lupus nephritis.

In particular, individuals suffering from systemic lupus erythematosus (SLE) often also bear lupus nephritis or start to develop lupus nephritis in later stages of the disease. SLE is an autoimmune disease which occurs comparably frequently. It is assumed that approximately 3.1 millions of individuals suffer from SLE worldwide (Rees et al., 2017, The worldwide incidence and prevalence of systemic lupus erythematosus: a systematic review of epidemiological studies, Rheumatology 56:1945-1961). Notably, it was observed that in approximately 50% of the individuals suffering from SLE also suffer from lupus nephritis (Bertsias et al., 2012, Joint European League Against Rheumatism and European Renal Association—European Dialysis and Transplant Association (EULAR/ERA-EDTA) recommendations for the management of adult and pediatric lupus nephritis, Annals of the Rheumatic Diseases 11:1771-1782; Davidson, 2016, What is damaging the kidney in lupus nephritis? Nature reviews, Rheumatology 12, 143-153).

SLE without the occurrence of lupus nephritis can be treated with rather mild agents, such as hydroxychloroquine, which rarely cause severe side reactions. In contrast, most cases of lupus nephritis necessitate treatment with a harsher immunosuppressive agents such as glucocorticoids in combination with other immunosuppressants such as cyclophosphamid, mycofenolate mofetil or calcineurin inhibitors (Kuhn et al., 2015, The Diagnosis and Treatment of Systemic Lupus Erythematosus. Deutsches Arzteblatt International 112:423-432). As many of these agents provoke severe undesired adverse reactions, it is of considerable interest to differentiate between SLE patients with and without lupus nephritis. Therefore, a differential diagnosis is of utmost importance.

This is, however, challenging. Many cases of lupus nephritis do not present with specific clinical symptoms. Diagnosis of lupus nephritis can only be made by means of an invasive intervention, i.e. biopsy of kidney tissue. It is evident that this intervention bears a critical health risk for the patient. Complications such as bleedings, formations of thrombi, etc. with the subsequent need for surgical interventions can occur. (Li et al., 2019, Imaging-Related Risk Factors for Bleeding Complications of US-Guided Native Renal Biopsy: A Propensity Score Matehing, Analysis, Journal of Vascular and Interventional Radiology: JV/R 30:87-94; Xu et al., 2017, Risk Factors for Severe Bleeding Complications in Percutaneous Renal Biopsy, The American Journal of the Medical Sciences 353:230-235). Also patient's compliance and psychological concerns might be an issue. Furthermore, it has been observed that, due to such invasive intervention, treatment of lupus nephritis is undesirably postponed until final pathological reports are available. Such delay can even result in a further increased morbidity. In extreme cases such postponement can even result in the patient's death due to kidney failure. (Ciruelo et al., 1996, Cumulative rate of relapse of lupus nephritis after successful treatment with cyclophosphamide, Arthritis and Rheumatism 39:2028-2034; Esdaile et al., 1994, The benefit of early treatment with immunosuppressive agents in lupus nephritis, The Journal of Rheumatology 21:2046-2051; Faurschou, et al., 2006, Prognostic factors in lupus nephritis: diagnostic and therapeutic delay increases the risk of terminal renal failure, The Journal of Rheumatology 33:1563-1569; Fiehn, et al., 2003, Improved clinical outcome of lupus nephritis during the past decade: importance of early diagnosis and treatment. Annals of the rheumatic diseases 62:435-439; Jacobsen et al., 1999; Prognostic value of renal biopsy and clinical variables in patients with lupus nephritis and normal serum creatinine. Scandinavian journal of rheumatology 28:288-299).

Accordingly, there is still an unmet need for non-invasive means for diagnosing lupus nephritis in an individual. Particularly desirably would be a biomarker for diagnosing lupus nephritis in an individual.

Surprisingly, it has been found that a protein that comprises an YB-1-like cold-shock domain which bears one or more guanidinylated lysinyl moieties (YB-1-G), is a biomarker that enables efficient diagnosis of lupus nephritis in an individual, in particular the individual suffering from systemic lupus erythematosus (SLE).

Accordingly, in a first aspect, the present invention relates to a method for diagnosing lupus nephritis in an individual suffering from systemic lupus erythematosus (SLE), said method conducted in vitro comprising identifying a protein that comprises an YB-1-like cold-shock domain of a sequence homology of at least 96% of SEQ ID NO: 14 which bears one or more guanidinylated lysinyl moieties (YB-1-G) in a sample S obtained from the individual.

According to the present invention, SEQ ID NO: 14 refers to the following polypeptide sequence:

DKKVIATKVLGTVKWFNVRNGYGFINRNDTKEDVFVHQTAIKKNNPRKY
LRSVGDGETVEFDVVEGEKGAEAANVTGPG.

The protein of interest may comprise the YB-1-like cold-shock domain of a sequence homology of at least 96% of SEQ ID NO: 14 in any position in its polypeptide sequence. It may be present at the or near by the N-terminus, at the or near by the C-terminus or anywhere else in the sequence of the protein.

The protein that comprises YB-1-G may also be designated as YB-1-G-containing protein.

In a further aspect, the present invention relates to a method for diagnosing lupus nephritis in an individual suffering from systemic lupus erythematosus (SLE), said method conducted in vitro comprising identifying an YB-1 protein, which bears one or more guanidinylated lysinyl moieties (YB-1-G-containing protein) in a sample S obtained from the individual.

The person skilled in the art will notice that the method of the present invention is preferably a method not directly associated with the diagnosis of the human or animal body. The result obtained by the method may be used for medical or non-medical purposes. This method allows an efficient differential diagnosis for SLE patients who potentially also have lupus nephritis.

Preferably, homology is determined according to the Basic Local Alignment Search Tool (BLAST) (as provided by the National Center for Biotechnology Information (NCBI) in October 2019).

An individual of interest (from which optionally an extracellular body fluid may be obtained from) may be a human or non-human animal. A non-human animal preferably is a non-human mammal, in particular a domestic mammal such as, e.g., a bovine, a pig, a horse, a donkey, a sheep, a camel, a goat, a dog, a cat, etc. Preferably, the individual of interest is a human. The individual, in particular when it is a human, may also be designated as patient or may be designated as subject. Thus, the terms “individual”, “subject” and “patient” may be understood exchangeably.

Preferably, the individual is suspected for having lupus nephritis, an increased propensity to develop lupus nephritis or being at risk of developing or having lupus nephritis.

As used herein, the term “lupus nephritis” may be understood in the broadest sense as a pathological health state designated as such. Typically, lupus nephritis is understood as an autoimmune disease that causes inflammation of one or both kidneys. Lupus nephritis typically is considered as a type of glomerulonephritis in which the glomeruli are inflamed. Preferably, lupus nephritis is the disease represented by the International Classification of Diseases (ICD-10) class M32.1 plus N08.5. It is typically associated with systemic lupus erythematosus (SLE), ICD codes under category M32.

The term “systemic lupus erythematosus” and its abbreviation “SLE” may be understood as an autoimmune disease in which the individual's immune system attacks healthy tissue in several parts of the individual's body. Common symptoms may include painful and swollen joints, fever, chest pain, hair loss, mouth ulcers, swollen lymph nodes, feeling tired, and a red rash. SLE may also be designated simplified as “lupus”. Preferably, SLE is the disease represented by the International Classification of Diseases (ICD-10) class M32.

As used herein, the terms “protein” and “polypeptide” may be understood interchangeably in the broadest sense as a compound mainly composed of natural amino acid moieties consecutively conjugated with another via amide bonds.

It will be understood that a protein in the sense of the present invention may bear the posttranslational modification of guanidinylation, e.g., of lysinyl moieties as already indicated above.

It will be understood that a protein in the sense of the present invention may or may not be subjected to one or more further posttranslational modification(s) and/or be conjugated with one or more non-amino acid moiety/moieties. The termini of the protein may, optionally, be capped by any means known in the art, such as, e.g., further guanidinylation, amidation, acetylation, methylation, acylation. Posttranslational modifications are well-known in the art and may optionally comprise lipidation, phosphorylation, sulfatation, glycosylation, truncation, oxidation, reduction, decarboxylation, acetylation, amidation, deamidation, disulfide bond formation, amino acid addition, cofactor addition (e.g., biotinylation, heme addition, eicosanoid addition, steroid addition) and complexation of metal ions, non-metal ions, peptides or small molecules and addition of iron-sulphide clusters. Moreover, optionally, co-factors, in particular cyclic guanidinium monophosphate (cGMP), but optionally also such as, e.g., ATP, ADP, NAD⁺, NADH⁺H⁺, NADP⁺, NADPH+H⁺, metal ions, anions, lipids, etc. may be bound to the protein, irrespective on the biological influence of these co-factors. It will be understood that such polypeptide may also bear one or more non-natural amino acid moiety/moieties and/or one or more posttranscriptional modification(s) and/or may be conjugated to one or more further structures such as label moieties (e.g., by means of a dye (e.g., a fluorescence dye) or a metal label (e.g., gold beads)). In the context of YB-1-G and a protein comprising YB-1-G (e.g., YB-1, DBPA/YB-3, etc.), the respective polypeptide is preferably the one generated by the individual of interest.

A lysinyl moiety may be understood as generally understood in the art as a residue derived from lysine, including a salt and a tautomer thereof, that is embedded in a polypeptide sequence. In the context of the present invention, typically and preferably, a lysinyl moiety has the following structure:

R¹—NH—CH((CH₂)₄—NH—R³)—CO—R² (1),

wherein:
residue R¹ is selected from the group consisting of a (poly)peptide moiety, an amino acid residue, hydrogen, an acyl residue (e.g., a acetyl residue), another residues present in (naturally occurring) polypeptides in this position, or a salt of any of the aforementioned, in particular R¹ is a polypeptide moiety;
residue R² is selected from the group consisting of a (poly)peptide moiety, an amino acid residue, —OH, —NH₂, another residues present in (naturally occurring) polypeptides in this position, or a salt of any of the aforementioned, in particular R² is a polypeptide moiety; and
residue R³ is selected from the group consisting of hydrogen, —C(NH)NH₂, another residues present in (naturally occurring) polypeptides in this position, or a salt or tautomer of any of the aforementioned, in particular R³ is hydrogen (unmodified lysinyl moiety) or —C(NH)NH₂ (guanidinylated lysinyl moiety) or a salt or tautomer thereof.

The terms “moiety”, “residue”, “rest” etc. may be understood interchangeably in the broadest sense as generally understood in the art.

According to the present invention, the YB-1-like cold-shock domain which bears one or more guanidinylated lysinyl moieties (YB-1-G) has a sequence homology of at least 96% of SEQ ID NO: 14. In a preferred embodiment, YB-1-G has a sequence homology of at least 97%, more preferably of at least 98%, even more preferably of at least 99% of SEQ ID NO: 14. In a particularly preferred embodiment, YB-1-G has (exactly) a sequence of SEQ ID NO: 14.

In a preferred embodiment, YB-1-G (having a polypeptide having a sequence homology of at least 96% of SEQ ID NO: 14) is guanidinylated in one or both of amino acid positions Lys3 and/or Lys8. In a preferred embodiment, YB-1-G (having a polypeptide having a sequence homology of at least 96% of SEQ ID NO: 14) is guanidinylated in both amino acid positions Lys3 and Lys8.

In a preferred embodiment, YB-1-G bears two guanidinylated lysinyl moieties, in particular at positions Lys3 and Lys8.

In a preferred embodiment, the method of the present invention comprises the following steps:
(i) providing the sample S (preferably (previously) obtained) from the individual, in particular a sample S of the extracellular body fluid; and
(ii) identifying YB-1-G in the sample S.

In a preferred embodiment, the method of the present invention comprises the following steps:
(i) providing the sample S (preferably (previously) obtained) from the individual, in particular a sample S of the extracellular body fluid; and
(ii) detecting YB-1-G in the sample S.

The “sample S” may be any sample from the individual of interest which may potentially comprise a protein containing YB-1-G. The designation “S” is only added as a name of the sample obtainable from the individual for clarity purposes to emphasize that a specific sample is meant. This designation can also be omitted without changing the scope, i.e., replacing “sample S” by “sample”. The sample S typically is an in vitro specimen, i.e., a specimen remote from the human and animal body, or is derived therefrom.

In a preferred embodiment, the sample S is liquid or viscous. In a preferred embodiment, the sample S may be an extracellular body fluid which is obtainable from the individual of interest. Or the sample S may comprise an extracellular body fluid or a fraction of an extracellular body fluid that may contain YB-1-G. In the sample S, an extracellular body fluid may optionally be diluted, e.g., in a buffer. In the context of the present invention, preferably all embodiments conductible with an extracellular body fluid can also be conducted with the sample S. Likewise, preferably all embodiments conductible with the sample S can also be conducted with an extracellular body fluid. The extracellular body fluid (also: the sample S consisting thereof or containing it) may be obtained by any means. Preferably, it is obtained by obtaining a blood sample of the individual of interest of which the blood cells and the clotting factors have been removed. The method of the present invention may be conducted in fresh blood serum (e.g., by contacting an ELISA or via a dipstick with the extracellular body fluid in a vessel/hollow ware) or may be conducted using a stored extracellular body fluid or an extracellular body fluid obtained from stored blood. Storage may be storage of up to 15 min (minutes), up to 30 min, up to one hour, up to twelve hours, up to a day, up to a week, up to a month, up to a year or even longer. Long-term storage for more than one day is preferably conducted under any conditions maintaining suitability of identifying YB-1-G (e.g., detecting of YB-1-G or detecting an anti-YB-1-G-autoantibody) such as, e.g., by means of freezing, shock-freezing (e.g., in liquid nitrogen), freeze-draying, and/or the addition of one or more preservative agents, in particular biocide/antimicrobial agents, to the extracellular body fluid.

In principle, the guanidinylated YB-1-like cold-shock domain (YB-1-G), which may be optionally comprised in a protein like an YB-1protein (YB-1), may be guanidinylated at any position, in particular at any lysinyl moiety/moieties. In a preferred embodiment, YB-1-G bears two guanidinylated lysinyl moieties (and may also be designated as YB-1-2G). In a preferred embodiment, YB-1-G bears two guanidinylated lysinyl moieties in the same domain of an YB-1 protein. In a preferred embodiment, the one or more guanidinylated lysinyl moieties are both located in the cold-shock domain (CSD) of an YB-1 protein. In a preferred embodiment, the two guanidinylated lysinyl moieties are (both) located in the cold-shock domain of an YB-1 protein. Optionally, YB-1-G can also bear more than two guanidinylated lysinyl moieties. Optionally, an YB-1 protein can also bear more than two guanidinylated lysinyl moieties.

According to the present invention, the YB-1-like cold-shock domain which bears one or more guanidinylated lysinyl moieties (YB-1-G) may be comprised in any protein. In a preferred embodiment, the protein that comprises the YB-1-like cold-shock domain is an YB-1 protein (YB-1). In other words, the guanidinylated YB-1-like cold-shock domain (YB-1-G) forms part of an YB-1 protein.

Preferably, in the context of the present invention, YB-1 has a homology of at least 80%, more preferably at least 90%, even more preferably at least 95%, even more preferably at least 98%, even more preferably at least 99% of SEQ ID NO: 1, in particular is identical with SEQ ID NO: 1:

MSSEAETQQPPAAPPAAPALSAADTKPGTTGSGAGSGGPGGLTSAAPAG
GDKKVIATKVLGTVKWFNVRNGYGFINRNDTKEDVFVHQTAIKKNNPRK
YLRSVGDGETVEFDVVEGEKGAEAANVTGPGGVPVQGSKYAADRNHYRR
YPRRRGPPRNYQQNYQNSESGEKNEGSESAPEGQAQQRRPYRRRRFPPY
YMRRPYGRRPQYSNPPVQGEVMEGADNQGAGEQGRPVRQNMYRGYRPRF
RRGPPRQRQPREDGNEEDKENQGDETQGQQPPQRRYRRNFNYRRRRPEN
PKPQDGKETKAADPPAENSSAPEAEQGGAE

Such human YB-1 protein may also be considered as being as the protein having the National Center for Biotechnology Information (NCBI) Reference Sequence: NP_004550.2.

YB-1 protein may be also designated as “YB-1”, “YB-1 polypeptide”, “Y-box binding protein-1”, “Y-box protein-1”, “YBX1”, “Y Box Binding Protein 1”, “YB1”, “YB-1”, “Y-Box Transcription Factor”, “Y Box Protein”, “YBOX1”, “NSEP1”, “DBPB”, “DNA-binding protein B”, “CCAAT-Binding Transcription Factor I Subunit A”, “Nuclease Sensitive Element Binding Protein”, “Enhancer Factor I Subunit A”, “EFI-A”, “DNA-Binding P”, “BP-8”, “Byb1”, “CSDA2”, “CSDB”, “DBPB”, “MDR-NF1”, “NSEP-1”, “NSEP1”, “Major Histocompatibility Complex Class II Y Box-Binding Protein I”, “C79409”, or the like. These terms are interchangeably as used herein. YB-1 protein is also known as nuclease-sensitive element-binding protein 1.

It may be defined that the full-length YB-1 protein has the following structure (starting from the N-terminus):

variable N-terminal domain which may have transactivation functionality (alanine/proline-rich, amino acid moieties 1 to 50);
cold-shock domain (CSD) which may be DNA/RNA-binding (amino acid moieties 51 to 129); and
C-terminal domain (CTD) which contains basic and acidic repeats (B/A) and comprises a nuclear localization signal (NSL) and a cytoplasmic retention site (CRS) (amino acid moieties 130 to 324)

YB-1 may also be a truncated or elongated form which comprises a sequence having a homology of at least 80%, more preferably at least 90%, even more preferably at least 95%, even more preferably at least 96%, even more preferably at least 97%, even more preferably at least 98%, even more preferably at least 99% of SEQ ID NO: 1, in particular is identical with SEQ ID NO: 1 that is truncated or elongated by one, two, up to five, up to 10, up to 20, up to 30, up to 50, up to 100 or up to 200 amino acid moieties.

In an alternative preferred embodiment, the YB-1-G may be comprised in DBPA/YB-3, which is also designated as “DNA-binding protein A”, “Cold shock domain-containing protein A”, “Single-strand DNA-binding protein NF-GMB”. This protein is encoded by a gene which can be designated as “YBX3”, “CSDA” or “DBPA”.

In a preferred embodiment, YB-1-G comprises a guanidinylated sequence of SEQ ID NO: 13:

KVIATK,

more preferably wherein the sequence is guanidinylated in one or both of amino acid positions Lys1 and/or Lys6, in particular wherein the sequence is guanidinylated in amino acid positions Lys1 and Lys6.

In a preferred embodiment, the individual is a human. In a preferred embodiment, the individual is a human and YB-1-G is a protein that comprises a human YB-1-like cold-shock domain of SEQ ID NO: 14 which is guanidinylated in amino acid moiety positions Lys3 and Lys8.

In a preferred embodiment, the individual is a human and YB-1-G is comprised in human YB-1 protein of SEQ ID NO: 1 which is guanidinylated in amino acid moiety positions Lys53 and Lys58.

In a preferred embodiment, the sample S is:
(a) an extracellular body fluid, in particular wherein the extracellular body fluid is blood serum or a fraction thereof;
(b) a tissue sample; or
(c) a combination of (a) and (b).

In a preferred embodiment, the sample S is an extracellular body fluid or contains an extracellular body fluid which is obtainable/obtained from the individual. Then, the present invention relates to a method for diagnosing lupus nephritis in an individual suffering from systemic lupus erythematosus (SLE), said method conducted in vitro comprising identifying a protein that comprises an YB-1-like cold-shock domain of a sequence homology of at least 96% of SEQ ID NO: 14 which bears one or more guanidinylated lysinyl moieties (YB-1-G) in an extracellular body fluid obtained from the individual. As used in the context of the present invention, an extracellular body fluid (also: the sample S) may be any body fluid that occurs in the individual out of cells or which is produced and optionally secreted by the individual.

For example, the extracellular body fluid may be selected from the group consisting of blood or a fraction thereof (including blood serum or a fraction thereof), urine, lymph, saliva, prerespirative excretion, cerebrospinal liquid, bile, further excretion products of glands, and combination of two or more thereof. Preferably, the extracellular body fluid is selected from the group consisting of blood or a fraction thereof (including blood serum or a fraction thereof), urine, lymph, saliva. Preferably, the extracellular body fluid is blood or a fraction thereof (including blood serum or a fraction thereof) or urine. Preferably, the extracellular body fluid is blood or a fraction thereof. Preferably, the blood cells are (essentially) removed and blood serum is obtained. In a preferred embodiment, the extracellular body fluid is blood serum or a fraction thereof. Typically, a fraction of a sample S still contains YB-1-G. Preferably, fraction of blood serum may be blood plasma or a Cohn's fraction of blood serum or blood serum or blood plasma subjected to one or more freeze-thaw cycles.

In an alternative preferred embodiment, the sample S may be a tissue sample. The tissue sample may also be a solid tissue sample. Then, the solid sample may optionally be involved in histologic investigations. A tissue sample may be obtained from the individual by any means. For example, it may be obtained from biopsy. Then, optionally, the obtained tissue or a part thereof may be stained with a labelled antibody specific for YB-1-G or a combination of an (optionally unlabeled) antibody specific for YB-1-G and a (preferably labelled) antibody specific for this antibody. Such direct and indirect labelling by antibodies is described further herein. The tissue sample may optionally also be cut into slices (e.g., by means of a microtome). Labelling may optionally also performed in a tissue sample that may be subjected to microscopy. The tissue sample may be subjected to in situ labelling. This may allow information on the localization of a protein containing YB-1-G in the tissue sample. Alternatively or additionally, the quantity of the protein containing YB-1-G in the tissue sample may be determined.

As used herein, the term “tissue sample” may also embrace may be a sample containing one or more cells (e.g., a suspension of cells or an adherent cell culture). A sample containing one or more (isolated) cells may be obtained from the individual by any means. The cells may be obtained from a body fluid or may be obtained from a tissue sample. The cells may be suspension cells or may be adherent cells. The cells may be directly obtained from the individual, may be cultivated for few days (e.g., up to 30 days, designatable as primary cell culture) or may be cultivated for a longer time (e.g., more than 30 days, designatable as permanent cell culture). Localization and/or quantity of a protein containing YB-1-G in the tissue sample may be determined in one or more cells. For example, quantity may be determined by means of flow cytometry and/or fluorescence-activated cell sorting (FACS). This may optionally Involve staining with a labelled antibody specific for YB-1-G or a combination of an (optionally unlabeled) antibody specific for YB-1-G and a (preferably labelled) antibody specific for this antibody. Such direct and indirect labelling by antibodies is described further herein.

A tissue sample may also be a liquid tissue sample such as body fluid that contains cell s such as, e.g., blood, lymph or cerebrospinal liquid. These are concomitantly samples S that are also extracellular body fluids as described above.

Thus, it will be understood that a sample S may also be a combination of an extracellular body fluid and a tissue sample. For instance, blood contains extracellular liquid (blood serum or a fraction thereof) and cells and cell-like bodies (red blood cells, white blood cells, platelets) and may be considered as a tissue.

As noted above, in a preferred embodiment, the individual suffers from systemic lupus erythematosus (SLE).

In principle, a method as claimed does not need any control samples. On the one hand the experimenter conducting the method will soon recognize certain thresholds indicating the borderline between a sample indicating the presence of lupus nephritis (e.g., sample S+ as described herein) and a sample of the same species indicating the absence of lupus nephritis (e.g., sample S− as described herein). This value may be determined for each device used.

As used herein, the terms “borderline” and “threshold” may be understood interchangeably in the broadest sense as a value that separates two groups from another.

When measuring a large number of samples (S) concomitantly, today, the intrinsic “control” is the average of values. Those levels significantly deviating from the average indicate values of relevance, i.e., patient that bear special characteristics such as lupus nephritis. Such borderline may also be determined as the average (e.g., arithmetic mean) of a number of samples (S) of individuals of the same species comprising those individuals having lupus nephritis and those not having lupus nephritis. In this context, preferably, number of samples (S) of individuals comprises samples (S) of at least two, at least five or at least ten individuals having lupus nephritis and at least two, at least five or at least ten individuals not having lupus nephritis.

In order to improve comparability of different samples (S) with another, in other words to normalize the determined and to improve reproducibility of the measurements, the determined YB-1-G level determined may be compared to a reference value. Such reference value may be an internal control (i.e., a further control sample S(0) as described below measured under comparable conditions, preferably in the same test series) or may be a predetermined reference value typically but not necessarily obtained from one or more previous measurements conducted under comparable conditions.

In order to improve comparability of different samples S with another, in other words to normalize the determined results, the level YB-1-G determined in a sample S is preferably related to a reference value such as the sample volume, the total polypeptide content comprised in same sample (e.g., the sample S or a control sample S(0), S+ or S− as each described in the following) or the content of an intrinsic marker (e.g., albumin, transferrin and/or beta-actin) of known concentration naturally contained in the same sample (e.g., an extracellular body fluid (i.e., also the sample S) or a control sample S(0), S+ or S− as each described in the following). If related to the sample volume, the level indicates the concentration, i.e., the respective polypeptide per volume (e.g., mass of YB-1-G per volume of the sample S [ng/ml]). If related to the total polypeptide content, a relative ratio may be provided (e.g., mass of YB-1-G per mass of total polypeptide content of the sample S [pg/ng]). If related to the content of an intrinsic marker, a relative ratio may be provided (e.g., mass of YB-1-G per mass of transferrin [pg/pg] or the mass of YB-1-G per mass of beta-actin [pg/pg]) or the mass of YB-1-G per mass of albumin [pg/ng]). Preferably, the step of determining the YB-1-G level is determining the level of YB-1-G in relation to the total polypeptide content comprised in the respective sample.

In a preferred embodiment, the method of the present invention comprises the following steps:
(i) providing the sample S (preferably (previously) obtained) from the individual (preferably of an extracellular body fluid); and
(ii) determining YB-1-G in the sample S, wherein:
   (iia) the presence of YB-1-G in the sample S indicates the presence of lupus nephritis in the individual, or
   (iib) an increased level of YB-1-G in the sample S indicates the presence of lupus nephritis in the individual, wherein the level of YB-1-G determined in the sample S is optionally compared with
      (a) a predetermined reference value indicating the borderline between a sample S+ indicating the presence of a lupus nephritis and a sample S− indicating the absence of lupus nephritis; and/or
      (b) an YB-1-G level determined in a control sample S(0) obtained from a control individual of the same species not having lupus nephritis,
      wherein an YB-1-G level determined in the sample S obtained from the individual that is higher than the YB-1-G level borderline between sample S− and S+ and/or at least 50% higher than sample S(0) indicates the presence of lupus nephritis in the individual,
      wherein the YB-1-G level in each case is related to the total polypeptide content comprised in the respective sample.

In a preferred embodiment, the method of the present invention comprises the following steps:
(i) providing the sample S (preferably (previously) obtained) from the individual, in particular a sample of an extracellular body fluid from the individual;
(ii) determining the level of YB-1-G in the sample S; and
(iii) comparing the YB-1-G level determined in step (ii) with
   (a) a predetermined reference value indicating the borderline between a sample S+ indicating the presence of a lupus nephritis and a sample S− indicating the absence of lupus nephritis; and/or
   (b) an YB-1-G level determined in a control sample S(0) obtained from a control individual of the same species not having lupus nephritis,
      wherein an YB-1-G level determined in the sample S obtained from the individual that is higher than the YB-1-G level borderline between sample S− and S+ and/or at least 50% higher than sample S(0) indicates the presence of lupus nephritis in the individual,
      wherein the YB-1-G level in each case is related to the total polypeptide content comprised in the respective sample.

Instead of measuring a control sample such as sample S(0) or S− or S+, also the background level (e.g., measurement of water or buffer or air, etc.) may be used.

Instead of measuring a control sample such as sample S(0) or S− or S+, in particular when conducted in an assays comprising a high number of samples (e.g., a high-throughput assay), the level of YB-1-G in the sample S may also be compared with the average of samples. This average of samples taken from individuals of the same species may indicate the borderline between the presence and absence of lupus nephritis.

Preferably, in this context, the YB-1-G level borderline between sample S− and S+ is determined by determining the distribution of several samples S− and several samples S+ and performing the Gaussian distributions (Gaussian curves) thereof. Then, the borderline is the YB-1-G level where the Gaussian distributions (Gaussian curves) cross.

In a preferred embodiment, an YB-1-G level determined in the sample S obtained from the individual that is at least 50% higher, at least 60% higher, at least 70% higher, at least 80% higher, at least 90% higher, at least 2fold higher, at least 5fold higher, or at least 10fold higher than the YB-1-G level determined in sample S− indicates lupus nephritis, wherein the YB-1-G level is related to the total polypeptide content comprised in the respective sample.

In a preferred embodiment, an YB-1-G level determined in the sample S obtained from the individual that is at least 50% higher, at least 60% higher, at least 70% higher, at least 80% higher, at least 90% higher, at least 2fold higher, at least 5fold higher, or at least 10fold higher than the YB-1-G level determined in sample S(0) indicates lupus nephritis, wherein the YB-1-G level is related to the total polypeptide content comprised in the respective sample.

It will be understood that typically the sample S and the control samples S(0), S− and S+ are obtained from the same species. Typically, protein contents are normalized.

YB-1-G may be identified by any means. Identifying YB-1-G may be direct or indirect identification of YB-1-G. In a preferred embodiment, identifying YB-1-G includes or is detecting YB-1-G (direct identification of YB-1-G). In an alternative preferred embodiment, identifying YB-1-G includes or is detecting an autoantibody specific for YB-1-G (anti-YB-1-G antibody, indirect identification of YB-1-G). Also immune cells (in particular activated B cells) that produce autoantibodies specific for YB-1-G may be determined.

In a preferred embodiment, identifying YB-1-G is detecting YB-1-G by means of detecting the selective binding of an antibody or a fragment thereof to YB-1-G. Thus, in a preferred embodiment, detecting YB-1-G includes the selective binding of an antibody or a fragment thereof to YB-1-G.

In a preferred embodiment, detecting YB-1-G includes one or both of selected from the group consisting of:
(a) direct immunodetection comprising providing at least one labeled antibody or antibody fragment specific for YB-1-G, and enabling binding of said labeled antibody or antibody fragment to YB-1-G; and
(b) indirect immunodetection comprising providing at least one (preferably unlabeled) antibody or antibody fragment specific for YB-1-G and at least one labeled antibody or antibody fragment specifically binding to said (preferably unlabeled) antibody or antibody fragment specific for YB-1-G, and
   enabling the binding of the (preferably unlabeled) antibody or antibody fragment to YB-1-G and the binding of the labeled antibody or antibody fragment to said (preferably unlabeled) antibody or antibody fragment specific for YB-1-G.

As noted above, identifying YB-1-G may also include or may be detecting an autoantibody specific for YB-1-G (anti-YB-1-G autoantibody). As used herein, an autoantibody that is generated by an individual that comprises YB-1-G (inherently). The presence of one or more anti-YB-1-G autoantibodies identifies the presence of YB-1-G in the individual (indirectly).

In a preferred embodiment, detecting an autoantibody specific for YB-1-G (anti-YB-1-G autoantibody) includes one or both of selected from the group consisting of:
(a) providing at least one epitope of an antibody or antibody fragment specific for YB-1-G;
(b) contacting the sample S obtained from the individual which potentially comprises an autoantibody specific for YB-1-G with said epitope, optionally washing;
(c) contacting a labeled antibody or antibody fragment specifically binding to said autoantibody; and
(d) detecting the presence or absence of autoantibodies specific for YB-1-G in the sample S obtained from the individual.

An antibody or a fragment thereof to YB-1-G, including prepared outside the individual's body as well as autoantibodies specific for YB-1-G may have the following characteristics.

Preferred characteristics of an YB-1-G-specific antibody or antibody fragment are that it binds to YB-1-G with a higher affinity than to a respective domain having the same sequence as YB-1-G which is not guanidinylated, in particular not guanidinylated in amino acid moiety positions Lys53 and Lys58. Preferably, in the context of YB-1-G, the antibody or antibody fragment binds to YB-1-G with an at least 10-fold, even more preferably at least 100-fold, even more preferably at least 1000-fold higher binding affinity than to a respective domain having the same sequence as YB-1-G which is not guanidinylated, in particular not guanidinylated in amino acid moiety positions Lys53 and Lys58.

In a preferred embodiment, such YB-1-G-specific antibody or antibody fragment is specific for an epitope that comprises at least eight, at least 10, at least ten, at least 15, or 20 or more that 20 consecutive amino acid moieties that encompass a sequence that at a homology of at least 80% to an amino acid sequence encompassing amino acid moiety positions Lys53 and Lys58 of SEQ ID NO: 1.

As used in the context of the present invention, the term “antibody” may be understood in the broadest sense as any type of immunoglobulin or antigen-binding fraction or variant thereof known in the art. Exemplarily, the antibody of the present invention may be an immunoglobulin A (IgA), immunoglobulin D (IgD), immunoglobulin E (IgE), immunoglobulin G (IgG), immunoglobulin M (IgM), immunoglobulin Y (IgY) or immunoglobulin W (IgW). Preferably, the antibody is an IgA, IgG or IgD. More preferably, the antibody is an IgG. However, it will be apparent that the type of antibody may be altered by biotechnological means by cloning the gene encoding for the antigen-binding domains of the antibody of the present invention into a common gene construct encoding for any other antibody type.

In one preferred embodiment of the present invention, the YB-1-G-specific antibody is an autoantibody specific for YB-1-G.

The binding between the antibody and its molecular target structure (i.e., its antigen, e.g., YB-1-G) typically is a non-covalent binding. Preferably, the binding affinity of the antibody to its antigen has a dissociation constant (Kd) of less than 1 μM, less than 500 nM, less than 200 nM, less than 100 nM, less than 50 nM, less than 40 nM, less than 30 nM or even less than 20 nM.

Preferably, the binding affinity to YB-1-G is higher than to a respective domain having the same sequence as YB-1-G which is not guanidinylated, in particular not guanidinylated in amino acid moiety positions Lys53 and Lys58. In a preferred embodiment, the antibody or antibody fragment binds to the YB-1-G with a dissociation constant of not more than 20 nM and, preferably, to a respective domain having the same sequence as YB-1-G which is not guanidinylated, in particular not guanidinylated in amino acid moiety positions Lys53 and Lys58, with a dissociation constant of more than 20 nM.

The term “antibody” as used herein may also include what may be designated as an antibody variant (also: antibody mutant). As used in the context of the present invention, the terms “antibody variant” and “antibody mutant” may be understood interchangeably in the broadest sense as any antibody mimetic or antibody with altered sequence known in the art. The antibody variant may have at least 10%, at least 20%, at least 50%, at least 60%, at least 70%, at least 80%, at least 85%, at least 90% or at least 95% of the binding affinity of a corresponding antibody, i.e., bear a dissociation constant (Kd) of less than 10 μM, less than 1 μM, less than 500 nM, less than 200 nM, less than 100 nM, less than 50 nM, less than 40 nM, less than 30 nM or even less than 20 nM.

As used herein, the term “antibody fragment” may be understood in the broadest sense as any fragment of an antibody that still bears binding affinity to its molecular target (i.e., its antigen, e.g., YB-1-G). Exemplarily, the antibody fragment may be a fragment antigen binding (Fab fragment), Fc, F(ab′)2, Fab′, scFv, a truncated antibody comprising one or both complementarity determining region(s) (CDR(s)) or the variable fragment (Fv) of an antibody. Variable domains (Fvs) are the smallest fragments with an intact antigen-binding domain consisting of one VL and one VH. Such fragments, with only the binding domains, can be generated by enzymatic approaches or expression of the relevant gene fragments, e.g. in bacterial and eukaryotic cells. Different approaches can be used, e.g. either the Fv fragment alone or ‘Fab’-fragments comprising one of the upper arms of the “Y” that includes the Fv plus the first constant domains. These fragments are usually stabilized by introducing a polypeptide link between the two chains which results in the production of a single chain Fv (scFv). Alternatively, disulfide-linked Fv (dsFv) fragments may be used. The binding domains of fragments can be combined with any constant domain in order to produce full length antibodies or can be fused with other polypeptides and polypeptides. A recombinant antibody fragment is the single-chain Fv (scFv) fragment. Dissociation of scFvs results in monomeric scFvs, which can be complexed into dimers (diabodies), trimers (triabodies) or larger aggregates such as TandAbs and Flexibodies. The antibody may be a Fab, a Fab′, a F(ab′)2, a Fv, a disulfide-linked Fv, a scFv, a (scFv)2, a bivalent antibody, a bispecific antibody, a multispecific antibody, a diabody, a triabody, a tetrabody or a minibody.

As mentioned above, the term “antibody” may also include an antibody mimetic which may be understood in the broadest sense as organic compounds that, like antibodies, can specifically bind antigens and that typically have a molecular mass in a range of from approximately 3 kDa to approximately 25 kDa. Antibody mimetics may be, e.g., affibody molecules (affibodies), aptamers, affilins, affitins, anticalins, avimers, DARPins, Fynomers, Kunitz domain peptides, single-domain antibodies (e.g., VHH antibodies or VNAR antibodies, nanobodies), monobodies, diabodies, triabodies, flexibodies and tandabs. The antibody mimetics may be of natural origin, of gene technologic origin and/or of synthetical origin. The antibody mimetics may also include polynucleotide-based binding units. Optionally, the antibody may also be a CovX-body. Optionally, the antibody may also be a cameloid species antibody.

The antibody according to the present invention is preferably a monoclonal antibody, a chimeric antibody or a humanized antibody. Monoclonal antibodies are monospecific antibodies that are identical because they are produced by one type of immune cell that are all clones of a single parent cell. A chimeric antibody is an antibody in which at least one region of an immunoglobulin of one species is fused to another region of an immunoglobulin of another species by genetic engineering in order to reduce its immunogenicity. For example, murine VL and VH regions may be fused to the remaining part of a human immunoglobulin. A particularly preferred type of chimeric antibodies are humanized antibodies. Humanized antibodies are produced by merging the DNA that encodes the CDRs of a non-human antibody with human antibody-producing DNA. The resulting DNA construct can then be used to express and produce antibodies that are usually not as immunogenic as the non-human parenteral antibody or as a chimeric antibody, since merely the CDRs are non-human.

The antibody or antibody fragment, independent on its chemical nature, may optionally be dissolved in any medium suitable for storing said antibody such as, e.g., water, an aqueous buffer (e.g., a Hepes, Tris, or phosphate buffer (e.g., phosphate buffered saline (PBS)), an organic solvent (e.g., dimethyl sulfoxide (DMSO), dimethylformide (DMF)) or a mixture of two or more thereof. The antibody or variant thereof according to the present invention may be of any species or origin. It may bind to any epitope(s) comprised by its molecular target structure (e.g., linear epitope(s), structural epitope(s), primary epitope(s), secondary epitope(s), i.e., such of, e.g., YB-1-G). Preferably, the antibody or variant thereof may recognize the naturally folded molecular target structure or a domain or fragment thereof (e.g., YB-1-G in the environment of blood serum). The antibody or variant thereof may be of any origin an antibody may be obtained from such as, e.g., natural origin, a gene technologic origin and/or a synthetic origin. Optionally, the antibody may also be commercially available. The person skilled in the art will understand that the antibody may further comprise one or more posttranscriptional modification(s) and/or may be conjugated to one or more further structures such as label moieties or cell-penetrating peptides (CPPs). Optionally, the antibody or antibody fragment may be added to a support, particularly a solid support such as an array, bead (e.g. glass or magnetic), a fiber, a film etc. The skilled person will be able to adapt the antibody of the present invention and a further component to the intended use by choosing a suitable further component.

In a preferred embodiment, identifying YB-1-G is conducted by means selected from the group consisting of enzyme-linked immunosorbent assay (ELISA), immuno-electrophoresis, immuno-blotting, Western blot, SDS-PAGE, a lateral flow dipstick, affinity chromatography, flow cytometry, fluorescence-activated cell sorting (FACS), microscopy, and combinations of two or more thereof.

In a preferred embodiment, in particular when the sample S is an extracellular body fluid, identifying YB-1-G is conducted by means selected from the group consisting of enzyme-linked immunosorbent assay (ELISA), immuno-electrophoresis, immuno-blotting, Western blot, SDS-PAGE, a lateral flow dipstick, affinity chromatography, and combinations of two or more thereof.

Preferably such means are based on one or more YB-1-G-specific antibodies. Such YB-1-G-specific antibody may also be an autoantibody specific for YB-1-G.

In a preferred embodiment, identifying YB-1-G is conducted by means of an enzyme-linked immunosorbent assay (ELISA). The ELISA may optionally also be a sandwich ELISA.

In a preferred embodiment, ELISA includes the following steps:
(i) providing:
   (A) a sample S comprising or consisting of an extracellular body fluid obtained from an individual (potentially containing YB-1-G),
   (B) a plate coated with an antibody or antibody fragment specific for YB-1-G, and
   (C) at least one further component for detecting YB-1-G selected from the group consisting of:
      (C1) a labelled antibody or antibody fragment specific for YB-1-G,
      (C2) a (preferably unlabeled) antibody or antibody fragment specific for YB-1-G and a labelled antibody or antibody fragment specific for detecting said preferably unlabeled) antibody or antibody fragment specific for YB-1-G,
      (C3) a labelled antibody or antibody fragment specific for guanidinylated lysinyl moieties,
      (C4) a (preferably unlabeled) antibody or antibody fragment specific for guanidinylated lysinyl moieties and a labelled antibody or antibody fragment specific for detecting said preferably unlabeled) antibody or antibody fragment specific for guanidinylated lysinyl moieties,
      (C5) a labelled antibody or antibody fragment specific for YB-1, and
      (C6) a (preferably unlabeled) antibody or antibody fragment specific for YB-1 and a labelled antibody or antibody fragment specific for detecting said preferably unlabeled) antibody or antibody fragment specific for YB-1;
(ii) contacting the sample S with the a plate and optionally subsequently washing the plate and removing unbound ingredients;
(iii) contacting the plate treated according to step (ii) with an component for detecting YB-1-G and optionally subsequently washing the plate and removing unbound ingredients; and
(iv) detecting the presence or absence or titer of component for detecting YB-1-G bound to the plate and, thus, detecting the presence or absence or titer of YB-1-G in the extracellular body fluid obtained from an individual.

In an alternative preferred embodiment, ELISA includes the following steps:
(i) providing:
   (A) a sample S comprising or consisting of an extracellular body fluid obtained from an individual (potentially containing YB-1-G),
   (B) a plate coated with an antibody or antibody fragment specific for YB-1, and
   (C) at least one further component for detecting YB-1-G selected from the group consisting of:
      (C1) a labelled antibody or antibody fragment specific for YB-1-G,
      (C2) a (preferably unlabeled) antibody or antibody fragment specific for YB-1-G and a labelled antibody or antibody fragment specific for detecting said preferably unlabeled) antibody or antibody fragment specific for YB-1-G;
      (C3) a labelled antibody or antibody fragment specific for guanidinylated lysinyl moieties, and
      (C4) a (preferably unlabeled) antibody or antibody fragment specific for guanidinylated lysinyl moieties and a labelled antibody or antibody fragment specific for detecting said preferably unlabeled) antibody or antibody fragment specific for guanidinylated lysinyl moieties;
(ii) contacting the sample S with the a plate and optionally subsequently washing the plate and removing unbound ingredients;
(iii) contacting the plate treated according to step (ii) with an component for detecting YB-1-G and optionally subsequently washing the plate and removing unbound ingredients; and
(iv) detecting the presence or absence or titer of component for detecting YB-1-G bound to the plate and, thus, detecting the presence or absence or titer of YB-1-G in the extracellular body fluid obtained from an individual.

In a still further alternative preferred embodiment, ELISA includes the following steps:
(i) providing:
   (A) a sample S comprising or consisting of an extracellular body fluid obtained from an individual (potentially containing one or more anti-YB-1-G-autoantibodies),
   (B) a plate coated with an epitope which is specific for an anti-YB-1-G-autoantibody (preferably YB-1-G or a fraction thereof, in particular YB-1-G or a fraction as described above), and
   (C) at least one further component for detecting an anti-YB-1-G-autoantibody selected from the group consisting of:
      (C1) a labelled antibody or antibody fragment specific for the anti-YB-1-G-autoantibody of interest,
      (C2) a (preferably unlabeled) antibody or antibody fragment specific for the anti-YB-1-G-autoantibody of interest and a labelled antibody or antibody fragment specific for detecting said anti-YB-1-G-autoantibody of interest,
      (C3) a labelled antibody or antibody fragment specific for an antibody of the species of the individual, and
      (C4) a (preferably unlabeled) antibody or antibody fragment specific for an antibody of the species of the individual and a labelled antibody or antibody fragment specific for detecting said antibody of the species of the individual;
(ii) contacting the sample S with the a plate and optionally subsequently washing the plate and removing unbound ingredients;
(iii) contacting the plate treated according to step (ii) with an component for detecting the anti-YB-1-G-autoantibody and optionally subsequently washing the plate and removing unbound ingredients; and
(iv) detecting the presence or absence or titer of component for detecting the anti-YB-1-G-autoantibody bound to the plate and, thus, detecting the presence or absence or titer of the anti-YB-1-G-autoantibody in the extracellular body fluid obtained from an individual and, thereby, optionally identifying YB-1-G in the individual (indirectly).

In a preferred embodiment, ELISA includes the following steps: Plates used for this purpose may be washed (e.g., by phosphate buffered saline (PBS)) and incubated with serum of the individual of interest and subsequently again washed (e.g., by phosphate buffered saline (PBS)) in order to remove unbound protein. The plate may then be incubated with an YB-1-G-specific antibody (e.g., antibody as exemplified herein) and again washed (e.g., by phosphate buffered saline (PBS)) in order to remove unbound antibody. Then, a secondary antibody (e.g., such binding to the Fc part of the YB-1-G-specific antibody) and again washed (e.g., by phosphate buffered saline (PBS)) in order to remove unbound antibody. This secondary antibody may be conjugated to an enzyme (e.g., peroxide). Exemplarily, a 3,3′,5,5′-Tetramethylbenzidin (TMB solution) may be used for staining purposes. A respective enzymatic label will be chosen. The reacting may finally be stopped (e.g., TMB solution by means of addition of sulfuric acid). The reaction products may be detected at a respective wavelength (e.g., in case of a TMB, of 495 nm). For the quantification and control, a high control (e.g., a serum of an individual having lupus nephritis) and a low control (e.g., a serum of a healthy individual) be optionally loaded on the same plate.

In alternative preferred embodiment, identifying YB-1-G is detecting YB-1-G conducted by means of mass spectrophotometry (MS), optionally including fractionation of the YB-1-G in a quadrupole and/or ion trap (MS-MS), wherein mass spectrophotometry is optionally combined with liquid chromatography (LC-MS). Detecting YB-1-G is conducted by means of mass spectrophotometry (MS) may also include fractionation of the YB-1-G in a quadrupole and/or ion trap, and maybe combined with liquid chromatography (LC-MS-MS). Exemplified embodiments are described in the example section.

The outcome of the method of the present invention may have a significant influence on the further treatment strategy. It may however also be used for scientific purposes only.

Therefore, in a preferred embodiment, the method of the present invention may comprise the further step of treating or preventing the individual in which lupus nephritis has been diagnosed. Preventing may also be attenuation of the progression of lupus nephritis. T may be understood as to guide treatment in individuals with lupus nephritis.

In a preferred embodiment, the method of the present invention may comprise the further step of treating or preventing the individual in which lupus nephritis has been diagnosed, wherein said individual is administered with a sufficient amount of a compound suitable for treating or preventing lupus nephritis. In particular wherein said compound is an anti-inflammatory and/or immune suppressing compound.

Accordingly, the present invention further relates to compound suitable for treating or preventing lupus nephritis for use in a method for treating or preventing lupus nephritis in an individual, wherein lupus nephritis has been diagnosed in the individual by means of a method of the present invention.

Thus, a further aspect of the present invention refers to an anti-inflammatory and/or immune suppressing compound for use in a method for treating or preventing lupus nephritis in an individual, wherein lupus nephritis has been diagnosed in the individual by means of a method of the present invention.

It will be understood that all specifications, aspects and embodiments described in the context of the method for diagnosing lupus nephritis in an individual as described above may apply mutatis mutandis to the anti-inflammatory and/or immune suppressing compound for use and vice versa.

In other words, the present invention further relates to a method for treating or preventing lupus nephritis in an individual, wherein said individual is administered with a sufficient amount of a compound suitable for treating or preventing lupus nephritis, wherein lupus nephritis has been diagnosed in the individual by means of a method of the present invention. In particular wherein said compound is an anti-inflammatory and/or immune suppressing compound.

The compound suitable for treating or preventing lupus nephritis may be any compound having such function in the individual's, e.g., human's body. The person skilled in the art knows numerous of such compounds. Preferably, it is an anti-inflammatory or immune suppressing compound. The anti-inflammatory or immune suppressing compound may be any compound having such function in the individual's, e.g., human's body. The person skilled in the art knows numerous of such compounds.

In a preferred embodiment, the anti-inflammatory or immune suppressing compound is selected from the group consisting of glucocorticoids such as hydrocortisone, prednisolone or dexamethasone, cyclophosphamide, methotrexate, azathioprine or mycophenolatmofetil, calcineurin inhibitors such as cyclosporine, tacrolimus or pimecrolimus, anti-inflammatory drugs (NSAIDs) such as cyclooxygenase (COX) inhibitors or immunosupressant antibodies.

Alternatively or additionally, also further cytostatica, TOR inhibitors such as sirolimus (rapamycin) or everolimus, belatacept, fingolimode, fumaric acid dimethylestes, and/or opioids may be used for this purpose.

In a preferred embodiment, one or more glucocorticoids are used in combination with one or more of the above mentioned immunosuppressant agents.

As noted above, YB-1-G has been surprisingly found to be an efficient biomarker for diagnosing lupus nephritis. Accordingly, a further aspect of the present invention relates to the use of a protein that comprises an YB-1-like cold-shock domain of a sequence homology of at least 96% of SEQ ID NO: 14 which bears one or more guanidinylated lysinyl moieties (YB-1-G) as a biomarker for diagnosing lupus nephritis.

In a preferred embodiment, the present invention relates to the use of an YB-1 protein which bears one or more guanidinylated lysinyl moieties (YB-1-G) as a biomarker for diagnosing lupus nephritis.

It will be understood that all specifications, aspects and embodiments described in the context of the method for diagnosing lupus nephritis in an individual and the anti-inflammatory and/or immune suppressing compound for use as described above may apply mutatis mutandis to the use and vice versa.

As noted above, in a preferred embodiment, identifying YB-1-G is conducted by means of an enzyme-linked immunosorbent assay (ELISA). Accordingly, a further aspect of the present invention relates to an ELISA kit for conducting a method of any of claims 1 to 9, comprising:
(A) an antibody or antibody fragment specific for YB-1-G or an epitope that is specifically bound by an anti-YB-1-G-autoantibody;
(B) optionally an antibody or antibody fragment specifically binding to said antibody or antibody fragment specific for YB-1-G or to said anti-YB-1-G-autoantibody;
(C) optionally one or more suitable buffers for the one or more antibodies or antibody fragments or epitopes;
(D) optionally one or more ELISA plates; and
(E) optionally user instructions.

In a preferred embodiment, the present invention relates to an ELISA kit for conducting a method of the present invention:
(A) an (unlabeled or labeled) antibody or antibody fragment specific for YB-1-G;
(B) optionally an (unlabeled or labeled) antibody or antibody fragment specifically binding to said antibody or antibody fragment specific for YB-1-G (anti-YB-1-G-antibody);
(C) optionally one or more suitable buffers for the one or more antibodies or antibody fragments;
(D) optionally one or more ELISA plates; and
(E) optionally user instructions.

In a preferred embodiment, the present invention relates to an ELISA kit for conducting a method of the present invention:
(A) an epitope that is specifically bound by an anti-YB-1-G-autoantibody;
(B) optionally an (unlabeled or labelled) antibody or antibody fragment specifically binding to said anti-YB-1-G-autoantibody;
(C) optionally one or more suitable buffers for the one or more antibodies or antibody fragments or epitopes;
(D) optionally one or more ELISA plates; and
(E) optionally user instructions.

It will be understood that all specifications, aspects and embodiments described in the context of the method for diagnosing lupus nephritis in an individual and the uses as described above may apply mutatis mutandis to the ELISA kits and vice versa.

Such kit may preferably further comprise user instructions for carrying out the method of the present invention. User instructions included in kits of the invention may be affixed to packaging material or may be included as a package insert. While the instructions are typically written or printed materials they are not limited to such. Any medium capable of storing such instructions and communicating them to an end user is contemplated by this invention. Such media include, for example, computer media including, but are not limited to, electronic storage media (e.g., magnetic discs, tapes, cartridges, chips), optical media (e.g., CD ROM), and the like.

As indicated above, the method of the present invention can also be conducted by means of a dipstick analysis (lateral flow analysis).

Accordingly, a still further aspect of the present invention refers to a dipstick usable for the method of any of claims 1 to 10 comprising, placed in the direction of flow of the sample S from the individual, in particular a sample of an extracellular body fluid, on a carrier that is suitable for soaking the sample S, the following:
(0) an edge or segment suitable for soaking the sample S;
(1) optionally a stripe (1) comprising labeled YB-1-G-specific antibodies or antibody fragments or epitopes that is specifically bound by an anti-YB-1-G-autoantibody which are not immobilized and freely movable when the sample S passes through this stripe (1);
(2) a stripe (2) comprising immobilized YB-1-G-specific antibodies or antibody fragments or an epitope that is specifically bound by an anti-YB-1-G-autoantibody; and
(3) optionally a stripe (3) of immobilized antibodies or antibody fragments specifically binding the one or more components of stripe (1).

A preferred embodiment refers to a dipstick usable for the method of the present invention comprising, placed in the direction of flow of the sample S from the individual, in particular a sample of an extracellular body fluid, on a carrier that is suitable for soaking the sample S, the following:
(0) an edge or segment suitable for soaking the sample S;
(1) optionally a stripe (1) comprising labeled YB-1-G-specific antibodies or antibody fragments which are not immobilized and freely movable when the sample S passes through this stripe (1);
(2) a stripe (2) comprising immobilized YB-1-G-specific antibodies or antibody fragments; and
(3) optionally a stripe (3) of immobilized antibodies or antibody fragments specifically binding the labeled YB-1-G-specific antibodies or antibody fragments of stripe (1).

Another preferred embodiment refers to a dipstick usable for the method of the present invention comprising, placed in the direction of flow of the sample S from the individual, in particular a sample of an extracellular body fluid, on a carrier that is suitable for soaking the sample S, the following:
(0) an edge or segment suitable for soaking the sample S;
(1) optionally a stripe (1) comprising labeled YB-1-G-specific antibodies or epitopes that is specifically bound by an anti-YB-1-G-autoantibody which are not immobilized and freely movable when the sample S passes through this stripe (1);
(2) a stripe (2) comprising an epitope that is specifically bound by an anti-YB-1-G-autoantibody;
(3) optionally a stripe (3) of immobilized antibodies or antibody fragments specifically binding the components of stripe (1).

It will be understood that all specifications, aspects and embodiments described in the context of the method for diagnosing lupus nephritis in an individual and the uses as described above may apply mutatis mutandis to the ELISA kits and vice versa.

The immobilized YB-1-G-specific antibodies or antibody fragments of stripe (2) may be unlabeled or labelled. Preferably, these are unlabeled.

Accordingly, a dipstick according to the present invention (preferably usable for the method of the present invention) comprises at least, placed in the direction of flow of the blood serum, on a carrier that is suitable for soaking the sample S, the following:
(0) an edge or segment suitable for soaking the sample S; and
(2) a stripe (2) comprising immobilized YB-1-G-specific antibodies or antibody fragments.

In an alternative embodiment, a dipstick according to the present invention (preferably usable for the method of the present invention) comprises at least, placed in the direction of flow of the blood serum, on a carrier that is suitable for soaking the sample S, the following:
(0) an edge or segment suitable for soaking the sample S; and
(2) a stripe (2) comprising an epitope that is specifically bound by an anti-YB-1-G-autoantibody.

As used herein, the terms “dipstick”, “dip-stick”, “test strip”, “control strip”, “diagnostic/medical dipstick” may be understood interchangeably in the broadest sense as any device that is usable to test an extracellular body fluid (i.e., also: a sample S) (according to the lateral flow technique). In the context of the dipstick, the sample S is typically liquid, semi-liquid or liquefied so that it can be soaked by a carrier of the dipstick. Typically, the sample S comprises an aqueous liquid. Exemplarily, the sample S usable by the dipstick may be or may be derived from blood serum from an individual.

In particular if the dipstick lacks stripe (1), the sample S is preferably premixed with a labeled YB-1-G-specific antibody or antibody fragment or fragment thereof. The volume and molar ratios will be adapted accordingly in order to optimize binding efficiency.

The volume of the sample S (optionally diluted and/or premixed with a labeled YB-1-G-specific antibody or antibody fragment) added to the dipstick will be adapted to the size and material of the dipstick. Typical volumes for adding to a segment suitable for soaking the sample S are in the range of from 10 to 1000 μl, preferably 50 to 500 μl, in particular 75 to 300 μl, exemplarily (approximately 200 μl). Exemplarily, the carrier may be a (hydro) gel or a piece of paper board, and may be optionally film laminated. Typically, the dipstick will be stored in dry state and is moistened by the sample S. When conducting the method of the present invention by means of the dipstick, the edge or segment suitable for soaking the sample S (0) may be contacted with the sample S. This is preferably conducted long enough to enable the sample liquid to be soaked in the carrier of the dipstick. The other parts of the dipstick are preferably not directly contacted with the sample S.

It is preferably enabled that the sample S flows through the carrier of the dipstick at least until the stripes (1) (if present) and (2) and optionally (3) have been passed by the sample S or parts thereof.

According to a preferred embodiment, the sample S is of a first species and the antibodies or antibody fragments of each of stripe (1) (if present) or the antibodies or fragments used for premixing with the sample S (in particular if stripe (1) is not present) on the one hand and (2) and optionally (3) of the other hand are each of different species.

In a preferred embodiment, the immobilized (preferably unlabeled) antibodies or antibody fragments of stripe (3) specifically bind to the Fc fragment of the labeled YB-1-G-specific antibodies or antibody fragments of stripe (1) (if present) or premixed with the optionally diluted blood serum (in particular if stripe (1) is not present). Exemplarily, the YB-1-G-specific antibodies or antibody fragments which are not immobilized are (preferably monoclonal) antibodies. Then, the immobilized antibodies of stripe (3) may be (preferably monoclonal) antibodies directed against the Fc part of the antibodies provided in stripe (1) or premixed with the sample S and optionally one or more buffers (in particular if stripe (1) is not present).

The label may be a fluorescence label, a visible dye label or, particularly preferably, a (colloidal) gold label. Such (colloidal) gold may be added to an antibody or antibody fragment by any means, exemplarily by means of a GOLD Conjugation Kit.

When an YB-1-G-containing sample S of an individual having lupus nephritis, is added to the dipstick, upon flowing through the dipstick, the labeled YB-1-G-specific antibodies may bind to YB-1-G in the sample S, thereby forming an YB-1-G/antibody conjugate. This conjugate will then bind to the (preferably unlabeled) YB-1-G-specific antibodies of stripe.

When a sample lacking YB-1-G of a healthy individual is added to the dipstick, upon flowing through the dipstick, the labeled YB-1-G-specific antibodies will not form an YB-1-G/antibody conjugate. Therefore, the polypeptides comprised in the sample S will then pass by the stripe (2) without being bound and will pass through the dipstick until the stripe (3). In such dipstick, the ratio between signal intensity of the label in stripe (2) and (3) may indicate the presence or lupus nephritis in the individual the sample S has been obtained from. A higher (2):(3) ratio indicates higher probability of the presence of lupus nephritis in the individual, whereas a lower (2):(3) ratio indicates lower probability of the presence of lupus nephritis in the individual in the sense of the method of the present invention laid out above.

The premixing of the labeled YB-1-G-specific antibodies or antibody fragments may be followed by an incubation to allow and optimize binding of the YB-1-G-specific antibodies or antibody fragments to its molecular target YB-1-G. This may exemplarily be performed by incubating for 10 to 60 min at a temperature of from 2 to 25° C.

An antibody or antibody fragment specific for YB-1-G also bears special beneficial technical effects in view of the prior art.

Accordingly, a still further aspect of the present invention relates to an antibody or antibody fragment specific for YB-1-G.

In a preferred embodiment, the antibody or antibody fragment binds to the YB-1-G with a dissociation constant of not more than 20 nM. In a preferred embodiment, the antibody or antibody fragment binds to a respective domain having the same sequence as YB-1-G which is not guanidinylated, in particular not guanidinylated in amino acid moiety positions Lys53 and Lys58, with a dissociation constant of more than 20 nM. In a preferred embodiment, the antibody or antibody fragment binds to the YB-1-G with a dissociation constant of not more than 20 nM, and binds to a respective domain having the same sequence as YB-1-G which is not guanidinylated, in particular not guanidinylated in amino acid moiety positions Lys53 and Lys58, with a dissociation constant of more than 20 nM.

Preferably, the antibody or antibody fragment thereof specific for YB-1-G according to the present invention may be usable in at least one of the methods selected from the group consisting of enzyme-linked immunosorbent assay (ELISA), immuno-electrophoresis, immuno-blotting, Western blot, SDS-PAGE, a lateral flow dipstick, affinity chromatography, flow cytometry, fluorescence-activated cell sorting (FACS), microscopy, and combinations of two or more thereof.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “contain”, “include” and “encompass” are to be interpreted inclusively rather than exclusively. Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, some exemplified preferred methods and materials are described herein.

The following Examples as well as the accompanying Figures are intended to provide illustrative embodiments of the present invention described and claimed herein. These Examples and Figures are not intended to provide any limitation on the scope of the invented subject-matter.

### BRIEF DESCRIPTION OF THE FIGURES

FIG. 1 shows that in lupus-prone mice and patients with SLE, serum YB-1 is guanidinylated at lysine residues 53 and 58, which correlates with disease progression and activates receptor Notch-3. (A) qRT-PCR analysis of mRNA levels for YB-1 in kidneys of 8, 12 and 20 week old MRL.lpr mice shows no significant changes during the time course of lupus (n=5-10). (B) Western blot analysis for YB-1 in kidney at 8, 12 and 20 weeks. Development of the blot with an anti-GAPDH antibody was used as confirmation for equal protein loading (n=3). (C/D) Western blot analysis for YB-1 in the serum of lupus mice compared to WT control (n=3) (C) and in human serum of healthy control individuals and SLE patients (D). (E-H) Guanidinylation of specific lysine residues in the cold-shock domain of YB-1 (E) in SLE patients (G) and not in controls (F) is shown by a characteristic mass fingerprint spectrum of tryptic digested YB-1. The arrow indicates the molecular mass of the peptide after tryptically digested YB-1 modified by guanidinylation. (H) The amino acid sequence of the peptide of interest is KVIATK, asterisks indicate the modifications. (I, J) Correlation of YB-1 guanidinylation status segregated with higher lupus activity scores SLEDAI (I) and BILAG (renal) (J). (K, L) qRT-PCR analysis of Notch target genes Hes1 (K) and Hes2 (L) in HUT78T cells transfected with Notch-3 plasmid and stimulated for 24 h with 10 ng/ml of either recombinant or guanidinylated YB-1 (YB-1-G-CONTAINING PROTEIN) (n=6-8). All in vitro experiments were performed at least in duplicates and repeated 3-4 times. Data are expressed as means±SD. n.s.=non-significant, *P<0.05, ***P<0.001, ****P<0.0001.

FIG. 2 shows that Notch-3 is upregulated as lupus progresses. (A) Renal mRNA expression of Notch1 and Notch3 receptors, and (B) mRNA expression of Notch target genes Hes2 and Hes5 during lupus progression in MRL.lpr mice (n=4-9). (C) Gene expression of Notch3 and Hes2 in spleens of MRL.lpr mice (n=4-9). (D) Heat map of mass spectrometric analysis of Notch-3 in kidney slices from 8 and 20 week old MRL.lpr mice. (E) Immunofluorescence staining of Notch-3 in kidneys of 8 and 20 week old MRL.lpr mice. Arrow shows Notch-3 positivity in the mesangium. (F, G) Immunofluorescence staining of Notch-3 and CD44+ by activated parietal cells (F) and of CD45+ immune cells (G) in kidneys of 20 week old MRL.lpr mice. Arrows indicate colocalization. Shown are representative images of one animal from a total of at least four animals analyzed per group. (H) Representative glomerular Notch-3 and CD44 immunofluorescence staining in a LN patient. (I) Quantification of the intensity of glomerular Notch-3 staining in human kidney biopsies of control individuals, IgAN and LN patients. Scale bars, 50 μm or as indicated. IgAN, IgA nephropathy, LN, lupus nephritis.

FIG. 3 shows that YB-1-G-CONTAINING PROTEIN is present in the diseased kidney. (NB) Heat map of mass spectrometric analyses of renal sections on YB-1 (A) and YB-1-G-CONTAINING PROTEIN (B) in 8 and 20 week old MRL.lpr mice. (C) Representative images of mass spectrometric analyses of renal sections for YB-1-G-CONTAINING PROTEIN, YB-1 and Notch-3 in MRL.lpr mice at 8 and 20 weeks of one animal from a total of four animals analyzed per group. (D) qRT-PCR analysis of Notch3 expression in primary parietal cells from WT, MRL.lpr and B16.lpr mice. (E) Notch3 mRNA expression of primary parietal cells from WT and B16.lpr mice stimulated with YB-1-G-CONTAINING PROTEIN (10 ng/ml; 24 h) compared to non-stimulated cells. All in vitro experiments were performed at least in duplicates and repeated 3-4 times.

FIG. 4 shows that Notch-3 deficiency aggravates lupus pathology in secondary lymphatic organs and kidneys. (A-C) mRNA transcripts of Notch3 (A/B) and Notch1 (C) were quantified in kidneys and spleens of B6, Notch3−/− (N3−/−), B6.lpr and B6.lprxNotch3−/− (B6.lprxN3−/−) mice by qRT-PCR and normalized to 18S rRNA content (n=4-11). (D) Survival rate of N3−/−, B6.lpr and B6.lprxN3−/− mice (n=16-64). (E) Images of lymph nodes, spleen and kidneys of B6.lpr and B6.lprxN3−/− animals. (F/H) HE-stained cross sections of spleens (F) (n=9-12) and lymph nodes (H) (n=3-9) from different genotypes and quantification of their respective areas (G/I). Data are expressed as means±SD. n.s.=non-significant, *P<0.05, **P<0.01, ****P<0.0001.

FIG. 5 shows that kidney damage and inflammation is aggravated in Notch-3 deficient lupus mice. (A) Serum creatinine levels, (B) cell proliferation in glomeruli, and (C/D) glomerular collagen IV deposition are increased in B6.lprxN3−/− (lprxN3−/−) in comparison to B6.lpr (lpr) animals. (E/F) Immunohistochemistry for CD45 in kidneys and quantification. (G) Gene expression of Notch-3 ligands Yb1 and Jagged as well as Notch-3 target gene Hes2 in kidneys of lprxN3−/− in comparison to lpr animals (n=8-11). Scale bars: 50 μm. Data are expressed as means±SD. n.s.=non-significant, *P<0.05, **P<0.01.

FIG. 6 shows dysbalance of immune cell subsets and impaired anti-inflammatory response in Notch-3 deficient lupus mice. (A-G) Percentages of different splenic immune cells from B6.WT, B6.lpr (lpr) and B6.lprxN3+/−(lprxN3+/−) animals determined by flow cytometry with the indicated markers: macrophages (A), dendritic cells (B), percentage of plasma cells within B cells (C), proportion of Th and cytotoxic T cells (D), and number of DN T cells within total T cells (E), percentage of activated T cells (F), percentage of regulatory Th cell subsets (G) and percentage of IL17+ cells (H) (n=4-5). (I) Renal IL17 protein amount of lprxN3−/− in comparison to lpr animals (n=6-7). (J/K) Splenic gene expression of Tgfβ (J) and Foxp3 in lprxN3−/− mice (K) (n=8-12). Data are expressed as means±SD. n.s.=non-significant, *P<0.05, **P<0.01, ***P<0.001, ****P<0.0001. Th, helper T cell; DN, double negative.

FIG. 7 shows that guanidinylated YB-1 activates Notch-3-dependent Il10 expression. (NB) qRT-PCR analysis of Notch target genes HES1, HES2 and IL10 in Notch-3 expression plasmid versus control vector-transfected HUT78T cells (n=6-7) (A) and non-transfected cells following TGFβ stimulation (5 ng/ml for 24 h) (n=5-8) (B). (C) Correlation of Notch3 and Il10 gene expression in spleens of 40-week-old B6.lpr mice (n=11). (D) Il10 gene expression in spleens and kidneys of B6.lpr and B6.lprxNotch3−/− mice (n=7-10). (E-H) Il10 (E), TGFb (F), IFNg (G) and Jagged (H) gene expression in Notch3 overexpressing HUT78T cells compared to control and stimulated for 24 h with 10 ng/ml recombinant YB-1 or its guanidinylated form YB-1-G-CONTAINING PROTEIN. (I) Notch3 and Il10 mRNA expression in transfected and YB-1-G-CONTAINING PROTEIN stimulated primary parietal cells isolated from WT mice. All in vitro experiments were performed at least in duplicates and repeated 3-4 times. Data are expressed as means±SD. n.s.=non-significant, *P<0.05, **P<0.01, ***P<0.001, ****P<0.0001.

FIG. 8 shows lupus activity scores in correlation to a single guanidinylation of YB-1 at lysine residue 58. Correlation of YB-1 guanidinylation status with the lupus activity scores SLEDAI (A) and BILAG (renal) (B) compared to healthy controls, with a score of 6 and A/B, respectively, indicating high activity.

FIG. 9 shows the localization of Notch-3 in glomeruli of 20 week old MRL.lpr mice. (A-F) Immunofluorescence staining for Notch-3 and (A) NG2 as a marker of pericytes (B) PDGFRb as a marker for mesenchymal cells, (C) Nestin as a marker for podocytes, (D) CD31 as a marker for endothelial cells, (E) CD45 as a marker for immune cells and (F) CD3 as a pan marker for T-cells. Shown are representative images of one animal from a total of at least four animals analyzed per group. Arrows indicate colocalization. (G) Representative glomerular Notch-3 immunofluorescence staining in a control individual, an IgAN, a DN and an FSGS patient. Scale bar 50 μm. IgAN, IgA nephropathy; DN, diabetic nephropathy; FSGS, focal segmental glomerular sclerosis.

### EXAMPLES

The experimental examples provide experimental evidence that YB-1-G-CONTAINING PROTEIN is a well-suitable biomarker for identifying lupus nephritis in an individual, in particular an individual suffering from systemic lupus erythematosus (SLE). Further, it is shown that the YB-1:Notch-3 axis modulates immune cell responses and organ damage in SLE (Manuscript accepted for publication in Kidney International.: Breitkopf et al.).

### Abstract

Systemic lupus erythematosus (SLE) is a systemic autoimmune disease and lupus nephritis bares a major risk for morbidity and mortality. Notch-3 signaling induced by membrane-bound or soluble ligands such as YB-1 constitutes an evolutionary conserved pathway that determines major decisions in cell fate. Mass-spectrometry of extracellular YB-1 in sera from SLE patients and lupus-prone mice revealed specific post-translational guanidinylation of two lysines within the highly conserved cold-shock domain of YB-1 (exemplary form of YB-1-G-CONTAINING PROTEIN, in the example section designated as “YB-1-G-CONTAINING PROTEIN”). These modifications highly correlated with SLE disease activity, especially in lupus nephritis patients and resulted in enhanced activation of Notch-3 signaling in T lymphocytes. The importance of YB-1:Notch-3 interaction in T cells was further evidenced by increased interleukin (Il)10 expression following YB-1-G-CONTAINING PROTEIN stimulation and detection of both, YB-1-G-CONTAINING PROTEIN and Notch-3, in kidneys of MRL. lpr mice by mass spectrometry imaging. Notch-3 expression and activation was significantly up-regulated in kidneys of 20-week-old MRL. lpr mice. Notably, lupus-prone mice with constitutional Notch-3 depletion (B6.Fas^{lpr/lpr}Notch3^{-/-}) exhibited an aggravated lupus phenotype with significantly increased mortality, enlarged lymphoid organs and aggravated nephritis. Besides, these mice displayed fewer regulatory T cells and reduced amounts of anti-inflammatory IL-10. Overall, inventors' results indicate that the YB-1:Notch-3 axis exerts protective effects in SLE and that Notch-3 deficiency exacerbates the SLE phenotype.

Notch signaling plays an essential role during kidney development and is largely silenced in the healthy adult kidney. Here, evidence for a significant up-regulation/activation of receptor homologue Notch-3 in kidneys from lupus-prone mice is provided as well as in biopsies from lupus nephritis patients. Furthermore, the inventors found the soluble Notch-3 ligand YB-1 to be expressed at increased levels and specifically guanidinylated (YB-1-G-CONTAINING PROTEIN) in sera from lupus mice and human patients with active lupus. Lupus-prone mice with genetic Notch-3 depletion demonstrated a profoundly increased mortality, enlargement of lymphoid tissues, aberrant immune cell populations and increased renal damage. The inventors' findings point to a protective interplay between YB-1-G-CONTAINING PROTEIN and Notch-3 in the development of lupus nephritis and therefore, Notch-3 activation through YB-1-G-CONTAINING PROTEIN might be a promising approach in the treatment of lupus patients.

### Introduction

Notch signaling constitutes an evolutionarily conserved pathway that transduces signals between neighboring cells, especially during organogenesis. The ancient receptor family of membrane-spanning glycoproteins includes four members (Notch-1 to Notch-4) in mammals¹. Once embryonic development is completed, Notch receptors are down-regulated, but may be reactivated in response to pathogenic stimuli. This is also true in renal inflammation/injury, which goes along with up-regulated Notch expression in the tubular and/or glomerular compartment^{2,3}. In particular, subtype Notch-3 is involved in acute and chronic kidney inflammation⁴. Consequently, genetic deletion of Notch-3 protected mice from tubulointerstitial fibrosis induced by ureteral obstruction².

Systemic lupus erythematosus (SLE) is a systemic autoimmune disease defined by immune dysregulation with loss of self-tolerance, autoantibody formation, vasculopathy and end organ damage. Organ involvement in SLE is variable; however, development of lupus nephritis (LN) is common and constitutes a serious organ- and life-threatening manifestation of SLE. Up to 10% of LN patients develop end-stage renal disease^{5,6}. Notch-1 expression in T cells affects SLE immunopathogenesis and Notch-1 transcript numbers correlate inversely with disease activity⁷. Few studies have specifically examined the role of Notch-3 in SLE.

The classical model of Notch receptor activation involves membrane-bound Notch ligands on adjacent cells, which are classified in mammals as either Delta-like (DII1, DII3 and DII4) or Jagged (Jag1 and Jag2). In addition, there are soluble non-canonical extracellular ligands. In contrast to the canonical ligands that all require binding to Notch extracellular domains, a consensus Notch binding site for non-canonical ligands has not been identified so far. In vertebrates secreted, non-canonical ligands include Y-box protein-1 (YB-1)⁴. Extracellular YB-1 is known to mediate inflammatory processes⁸⁻¹¹ and to activate Notch-3 signaling by binding to epidermal growth factor-like repeat 20-23¹². In a model of mesangioproliferative glomerulonephritis, YB-1 and Notch-3 are temporarily up-regulated in the glomeruli in a coordinated manner⁴. Herein, it was sought to clarify the role of Notch-3 and its soluble ligand YB-1 in SLE/LN and demonstrate that the presence of modified YB-1 and the activation of its receptor Notch-3 counteract the progression of SLE.

### Results

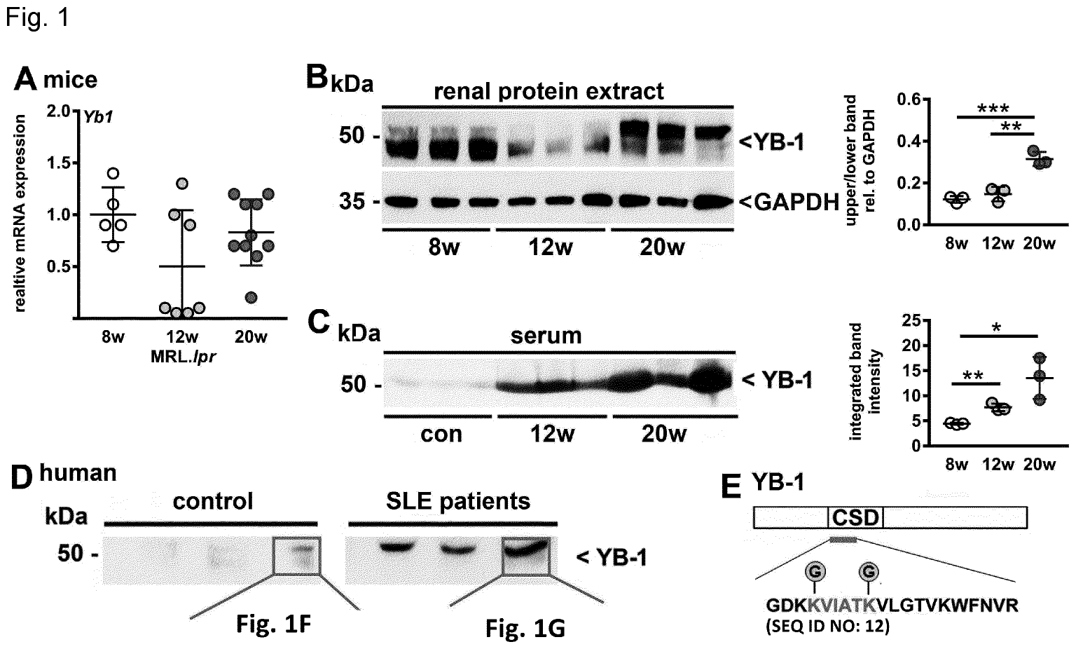

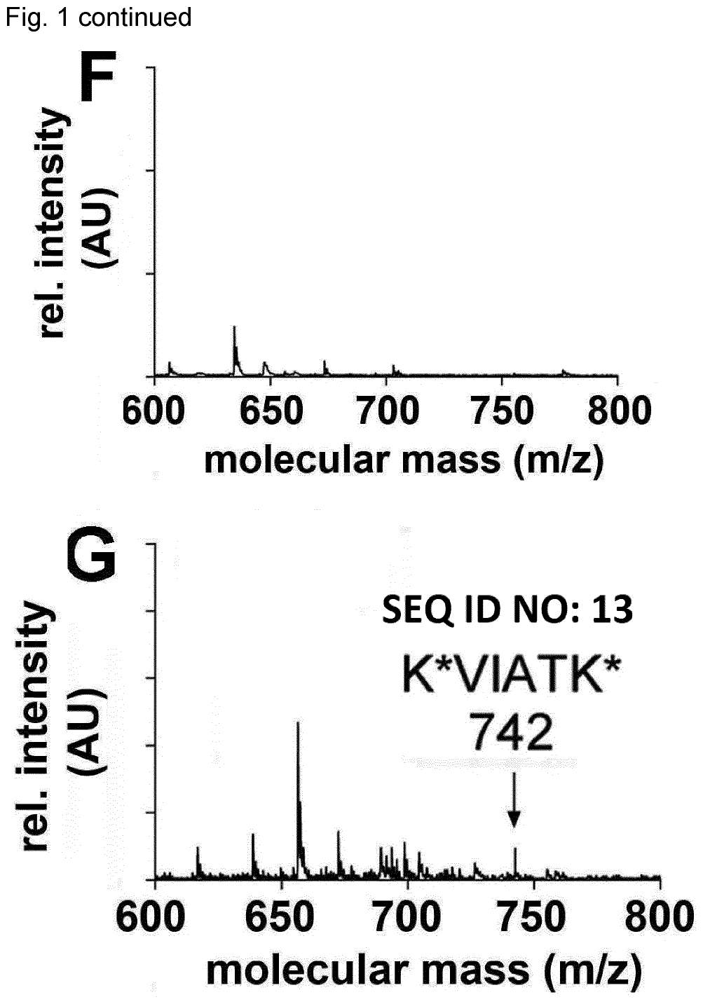

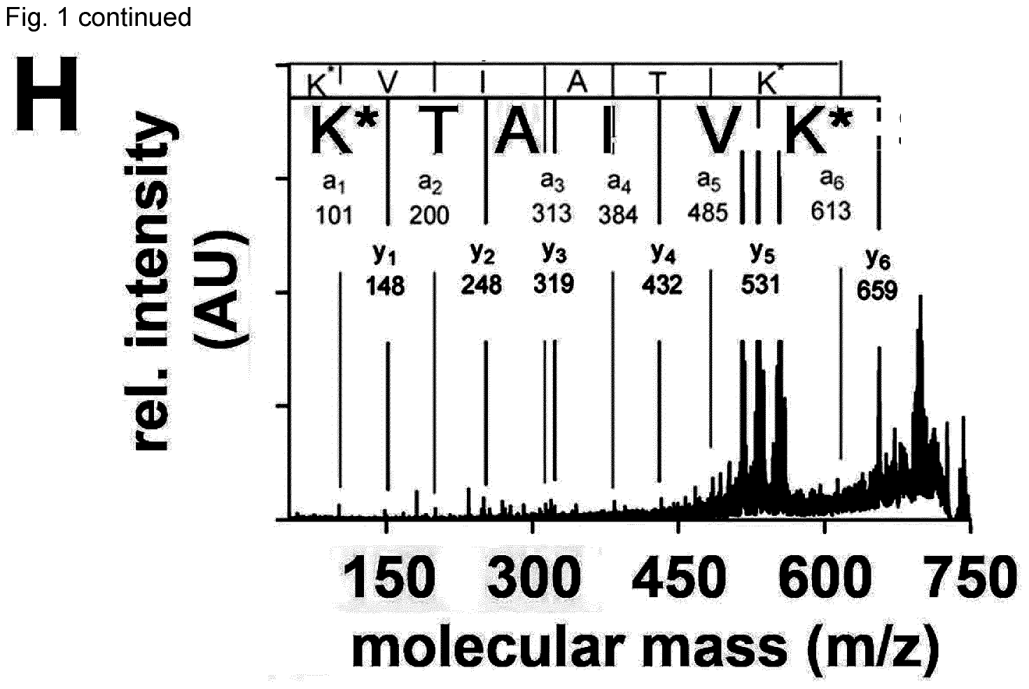

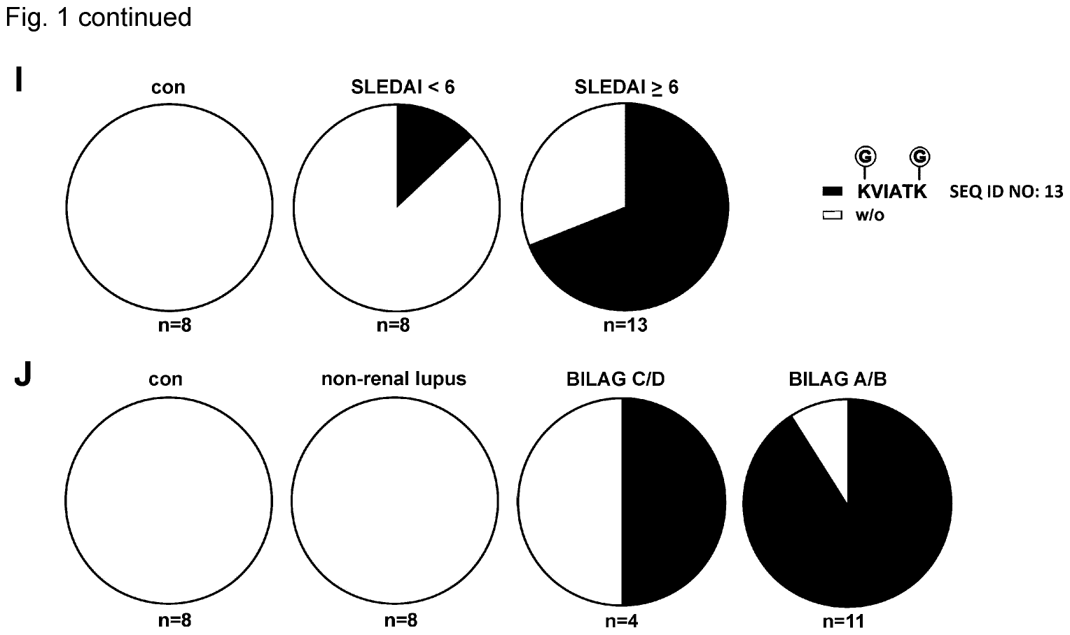

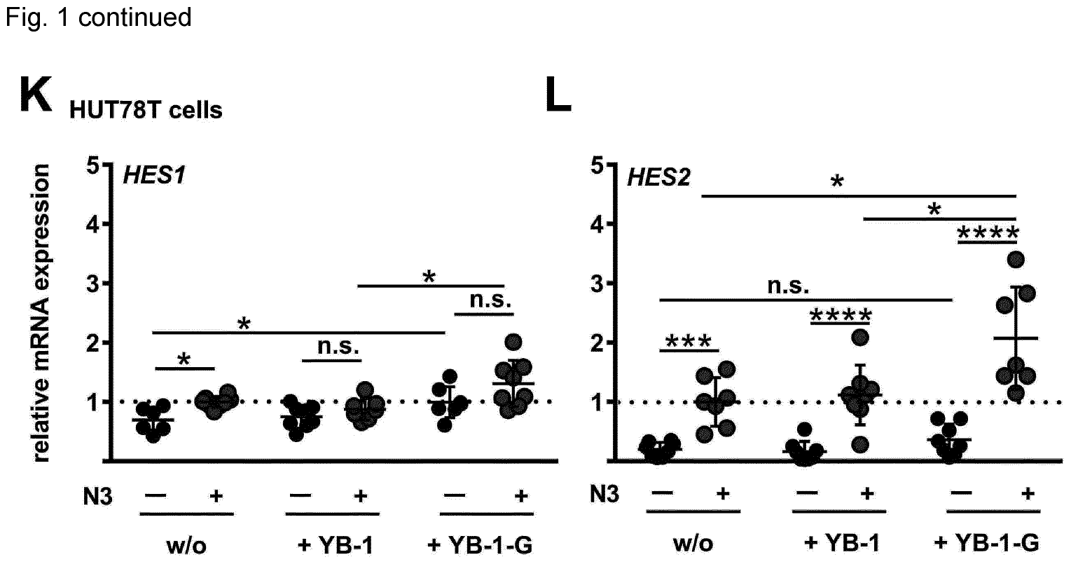

Sera from SLE patients contain higher levels of a guanidinylated YB-1 protein. It was first assessed the YB-1 content in renal cortices from MRL. lpr mice, which exhibit an autoimmune, lupus-like phenotype including development of a severe glomerulonephritis. Compared to young MRL. lpr mice without overt signs of lupus pathology at 8 weeks, renal YB-1 mRNA expression and protein content decreased at week 12 with a subsequent increase until week 20 when lupus manifestations had fully developed (FIGS. 1A/B). However, at week 20, it was observed a slightly higher molecular weight of the YB-1 protein (FIG. 1B). The upper to lower band ratio revealed significantly more high molecular weight YB-1 in the 20-week-old lupus animals (FIG. 1B, right bands). Serum levels of secreted YB-1 increased significantly over time and peaked in 20-week-old animals (FIG. 1C).

YB-1 was also detectable in sera from SLE patients as compared to healthy controls (FIG. 1D). Mass spectrometry of immunoblotted sera from MRL. lpr mice, SLE patients and healthy control individuals identified specific post-translational modifications in lupus mice (data not shown) and patients, i.e. guanidinylations at two lysine residues in close proximity (K53/K58) within the highly conserved cold-shock domain (CSD) of YB-1 (FIGS. 1E-H). In lupus patients, those with high disease activity (SLE disease activity index, SLEDAI 6) had a higher degree of YB-1 dual-guanidinylation as compared to SLE patients with lower disease activity and non-SLE controls (FIG. 1I) (Bombardier et al., 1992, Derivation of the SLEDAI. A disease activity index for lupus patients, The Committee on Prognosis Studies in SLE, Arthritis and Rheumatism 35:630-640). Ten out of 11 samples with active nephritis (renal BILAG NB) had YB-1 dual-guanidinylation whereas less or no dual-guanidinylation was observed in LN patients with inactive nephritis (renal BILAG C/D) and non-renal lupus patients (Murphy et al., 2016, From BILAG to BILAG-based combined lupus assessment—30 years on. Rheumatology 55:1357-1363).

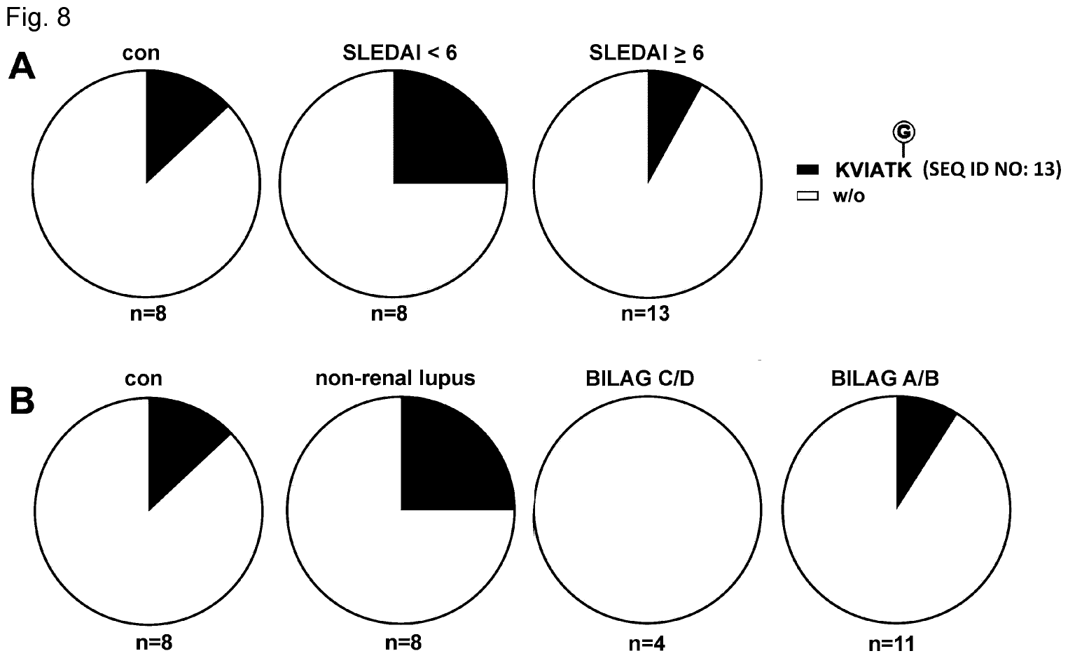

Furthermore, active SLE patients were more likely to display the dual than a single guanidinylation only at K58 (FIG. 8). Of note, at least one of these modifications (K53/K58 dual-guanidinylation or K58 single-guanidinylation) was present in all analyzed LN patients (n=11). Among the eight control individuals, single-guanidinylated YB-1 was present in only one person and nobody exhibited a dual-guanidinylation (FIGS. 1I/J, FIG. 8, left). Demographic and clinical patients' characteristics are given in Table 1.

TABLE 1
Demographic and clinical patents' data

| | SLEDAI < 6 | SLEDÄ1 6 |
|---|---|---|
| | (n = S) | (n = 13) |
| Female patients (%) | 88 | 80 |
| Age | 46.3 ± 16.5 | 24.0 ± 8.5 |
| SLEDAI | 1.6 ± 1.1 | 13.6 ± 6.8 |
| ANA positivity (%) | 100 | 100 |
| Patients with anti-dsDNA >100 kU/1 (%) | 25 | 19 |
| Serum C3 (g/1) | 0.90 ± 0.26 | 0.72 ± 0.35 |
| Serum C4 (g/1) | 0.14 ± 0.05 | 0.16 ± 0.08 |
| Serum creatinine (mg/dl) | 0.89 ± 0.14 | 0.94 ± 0.23 |
| eGFR (ml/min/1.73 m²) | 80.7 ± 20.8 | 86.5 ± 20.6 |
| Proteinuria (g/day} | 0.12 ± 0.05 | 2.58 ± 2.95 |
| Patients under immunosuppression (%) | 88 | 94 |

It was previously detected secreted YB-1 in sera of septic patients and attributed pro-inflammatory properties were attributed to extracellular YB-1 in murine sepsis models⁹. Since secreted YB-1 acts as a soluble, signaling ligand for membrane-bound Notch-3 receptors¹³, in vitro studies were performed in human T lymphocytes (HUT78T cells) with and without forced full-length Notch-3 receptor expression. When recombinant YB-1 protein was added to the cell culture medium, the Notch-3 target gene HES2 was significantly up-regulated. This effect was even more pronounced upon treatment with a modified, recombinant YB-1 protein harboring the dual-guanidinylation (K53/K58) observed in the lupus cohort. HES1 was activated by guanidinylated YB-1 only (FIGS. 1K/L).

Thus, a considerable amount of specifically guanidinylated YB-1 protein is present in sera from lupus-prone mice and patients with active SLE. Furthermore, secreted and particularly guanidinylated YB-1 activates Notch-3 receptor signaling.

### Notch-3 is Upregulated Upon Manifestation of LN and YB-1-G-CONTAINING PROTEIN is Present in the Diseased Kidney

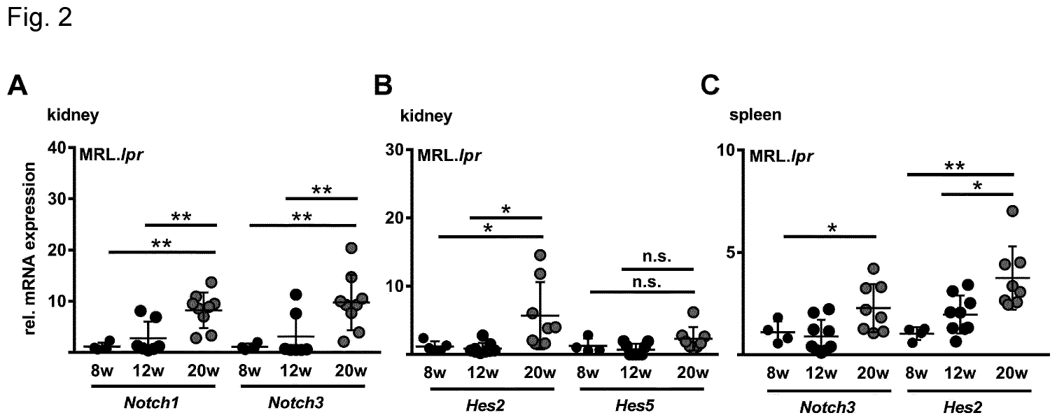

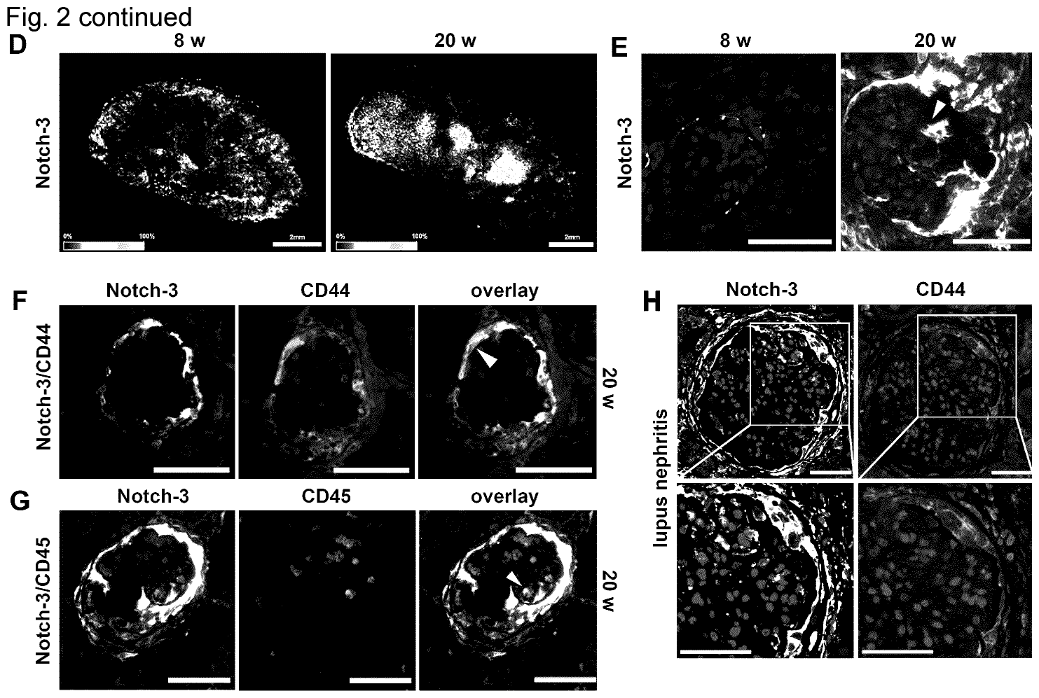

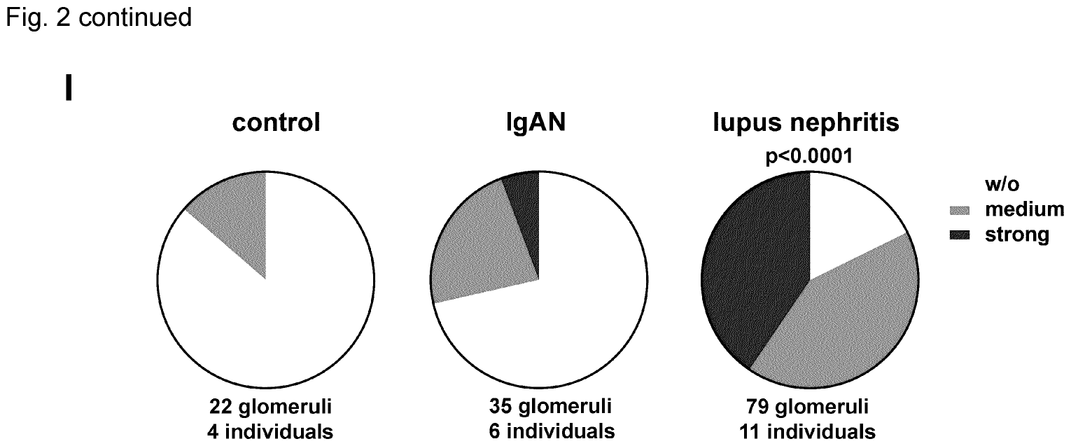

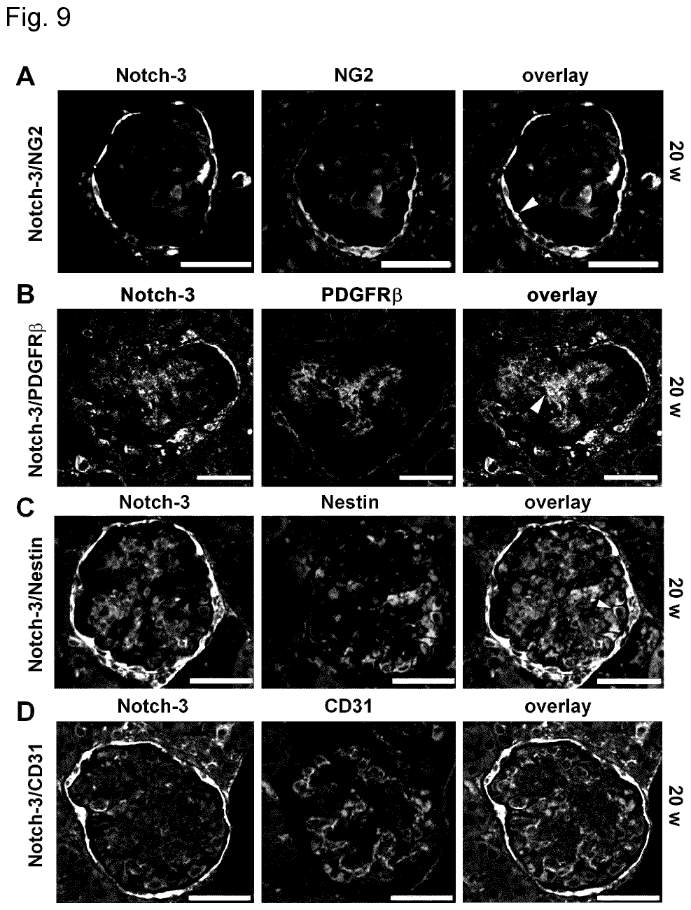

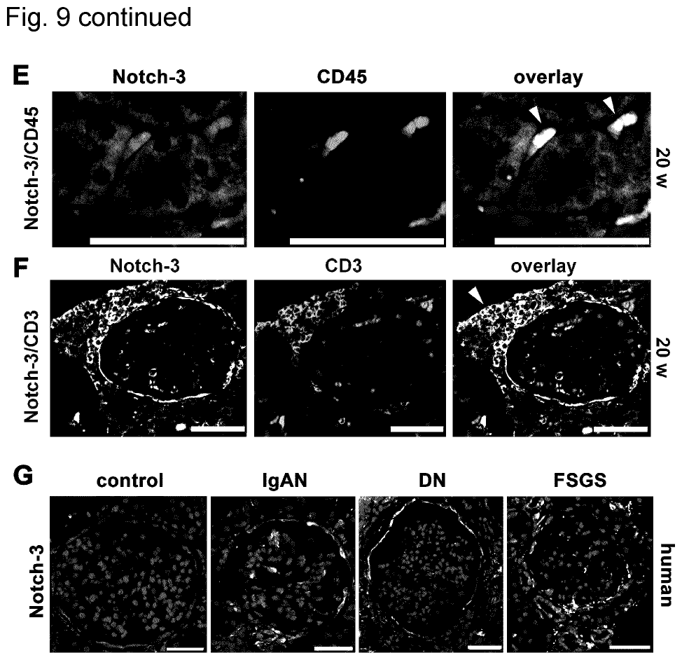

In MRL. lpr mice receptor homologues Notch 1 and Notch3 (FIG. 2A) as well as their target gene Hes2, but not Hes5, (FIG. 2B) were significantly up-regulated over time at mRNA levels. Furthermore, expression of Notch3 and its target gene Hes2 was increased in spleens of lupus mice (FIG. 2C), although not as pronounced as in the kidney. By mass spectrometry imaging (MALDI MSI) only small amounts of Notch-3 were detected in kidneys of 8-week-old MRL. lpr animals and this markedly increased in diseased, 20-week-old mice (FIG. 2D). Confirmed by immunofluorescence, Notch-3 protein was overexpressed upon lupus progression in resident glomerular (FIGS. 2E/F and FIGS. 9A-D) cells, infiltrating immune cells in glomeruli (FIG. 2G and FIG. 9F) and in the renal tubulointerstitium (FIG. 9E). CD44, a marker of activated glomerular parietal epithelial cells (PECs) (FIG. 2F), NG-2, a marker of pericytes (FIG. 9A), and PDGFR-beta (FIG. 9B), a marker of mesenchymal cells, partially co-localized with Notch-3 in 20-week-old MRL. lpr mice, whereas only minor co-staining for Notch-3 was observed with podocyte marker nestin (FIG. 9C) and with endothelial cell marker CD31 (FIG. 9D). It was observed CD3-positive Notch-3-positive immune cell infiltrates in close vicinity to some glomeruli (FIG. 9F).

Next, it was evaluated renal Notch-3 expression in patients with proliferative LN forms. Glomerular Notch-3 expression increased significantly in LN patients as compared to non-diseased controls and patients with IgA nephropathy (IgAN; FIG. 2I and FIG. 9G). In agreement with the murine data, Notch-3 partially co-localized with CD44 (FIG. 2H). In patients with diabetic nephropathy (DN) Notch-3 expression was detected. However, patients with focal-segmental glomerulosclerosis (FSGS) showed a primary Notch-3 expression in crescentic lesions (FIG. 9G).

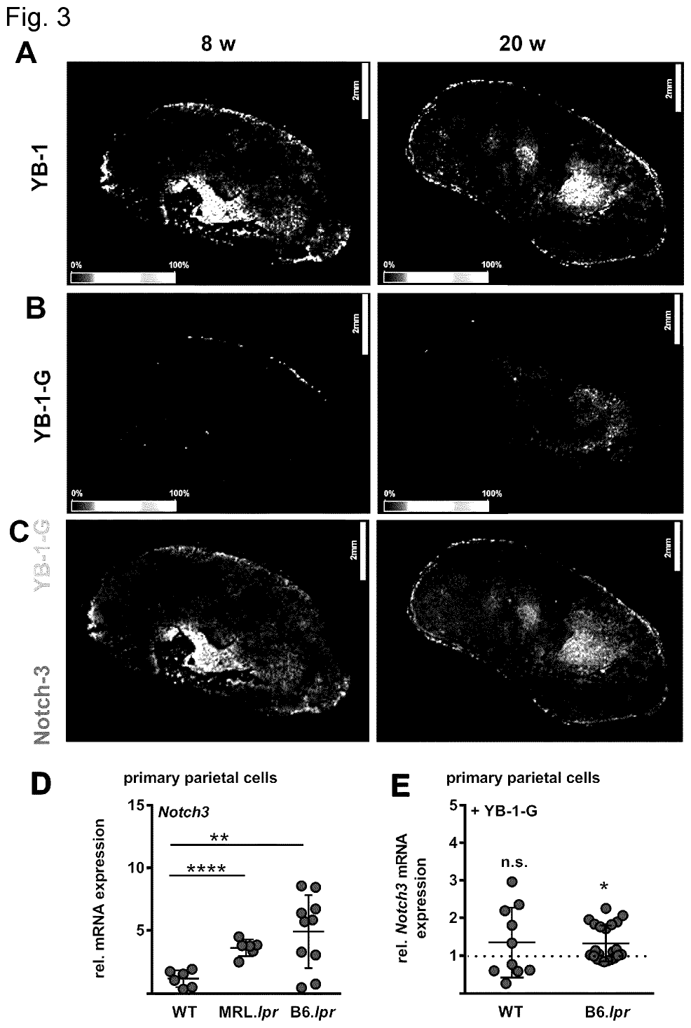

MALDI MSI was used to specifically localize YB-1-G-CONTAINING PROTEIN in kidney sections from 8- and 20-week old MRL. lpr mice. In comparison to overall YB-1 presence (FIG. 3A), the modified form was almost undetectable in young animals but clearly enhanced in diseased, 20-week old mice (FIGS. 3B and C). Enhanced Notch-3 expression in parietal cells in lupus mice was confirmed in isolated primary cells (FIG. 3D) and extracellular YB-1-G-CONTAINING PROTEIN induced Notch-3 expression in parietal cells of lupus-prone mice (FIG. 3E). In summary, it was demonstrated that renal Notch-3 is up-regulated in lupus mice and LN patients, in particular in the glomeruli, and that its modified ligand YB-1 can also be detected in diseased kidneys.

### Notch-3 Deficiency Aggravates Lupus Pathology in Secondary Lymphatic Organs and Kidneys.

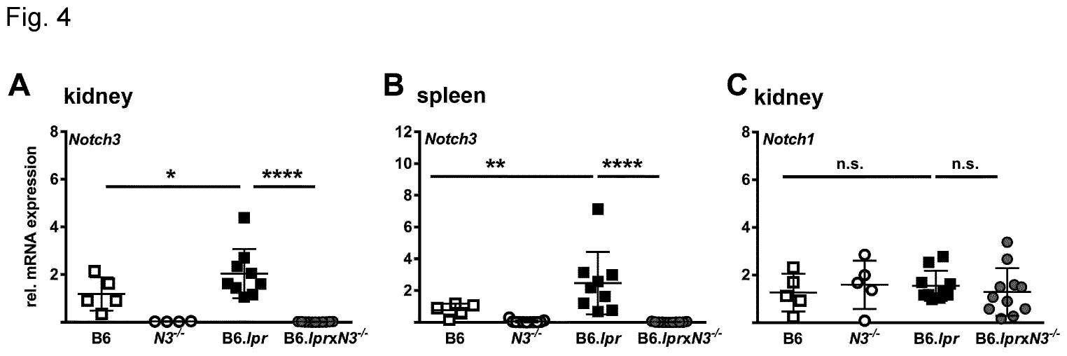

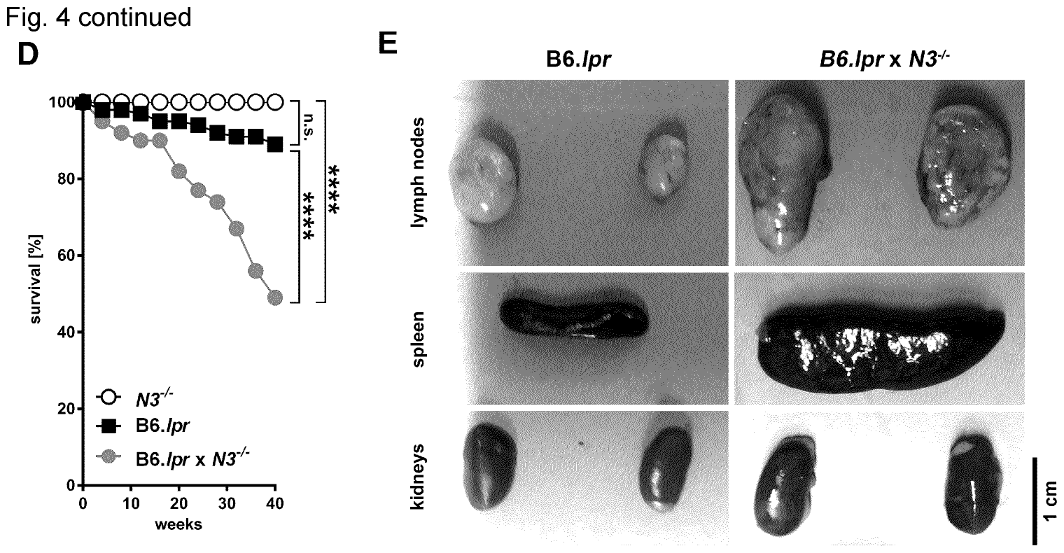

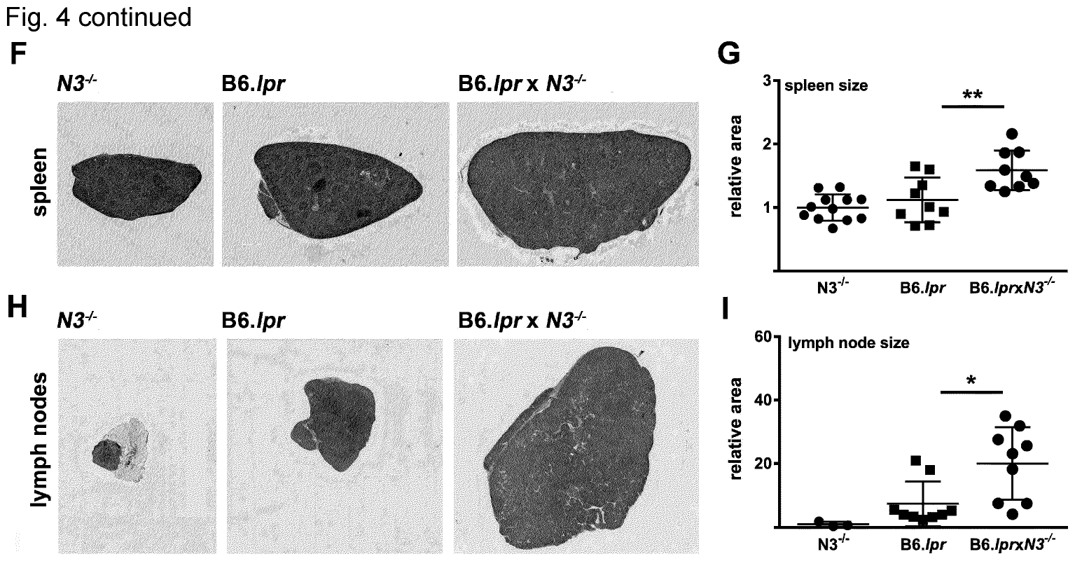

Next, endogenous Notch-3 levels were genetically depleted in mice bearing the lpr mutation on a B6 background. Compared to the MRL. lpr strain, B6.lpr mice develop lupus-like features to a milder extent. A Notch-3 knock-out mouse on the B6.lpr background (B6. lpr×N3^{-/-}) was generated and these mice were analyzed at an age of 40 weeks. As in the MRL. lpr strain, renal and splenic Notch3 mRNA expression was increased in 40-week-old B6.lpr mice (FIGS. 4A/B). Notch3 mRNA was undetectable in kidneys (FIG. 4A) and spleens (FIG. 4B) of B6. lpr×N3^{-/-} mice, whereas renal expression of receptor homologue Notch1 remained unaffected by genetic Notch3 depletion (FIG. 4C). B6.lpr×N3^{-/-} mice exhibited markedly increased mortality (51% at week 40) as compared to B6.N3^{-/-} (0%) and B6.lpr (11%) mice (p<0.0001 for both comparisons; FIG. 4D). Secondary lymphatic organs such as peripheral lymph nodes and spleens were enlarged in B6. lpr×N3^{-/-} mice as compared to B6.lpr mice (FIGS. 4E-I).

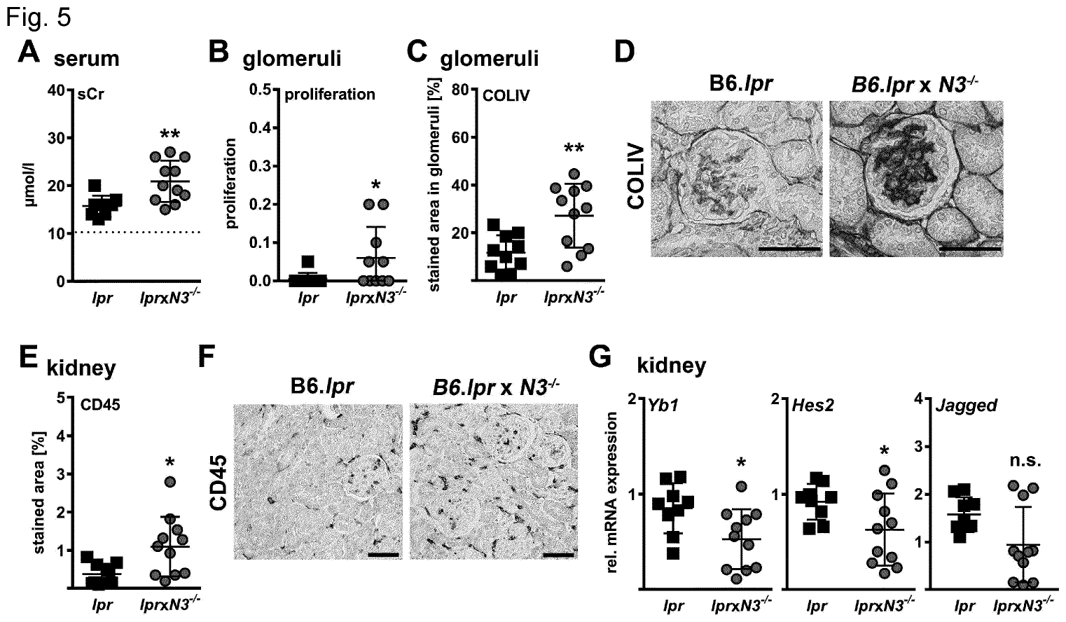

40-week-old B6.lpr mice displayed increased serum creatinine levels compared to controls (FIG. 5A, dashed line), and these further increased in B6.lpr×N3^{-/-} mice (FIG. 5A). Along these lines, it increased glomerular cell proliferation was noted (FIG. 5B), more pronounced glomerular collagen IV deposition (FIGS. 5C/D) and more extensive renal immune cell infiltration (FIGS. 5E/F) in B6.lpr×N3^{-/-} as compared to B6.lpr mice. In kidneys obtained from B6.lpr×N3^{-/-} animals, mRNA expression of both, the soluble ligand Yb1 and the Notch-3 target gene Hes2, was reduced as compared to B6.lpr mice, whereas renal expression of the canonical ligand Jagged only showed a tendency towards lower expression levels (FIG. 5G).

### Dysbalance of Immune Cell Subsets and Impaired Anti-Inflammatory Response in Notch-3-Deficient Lupus Mice.

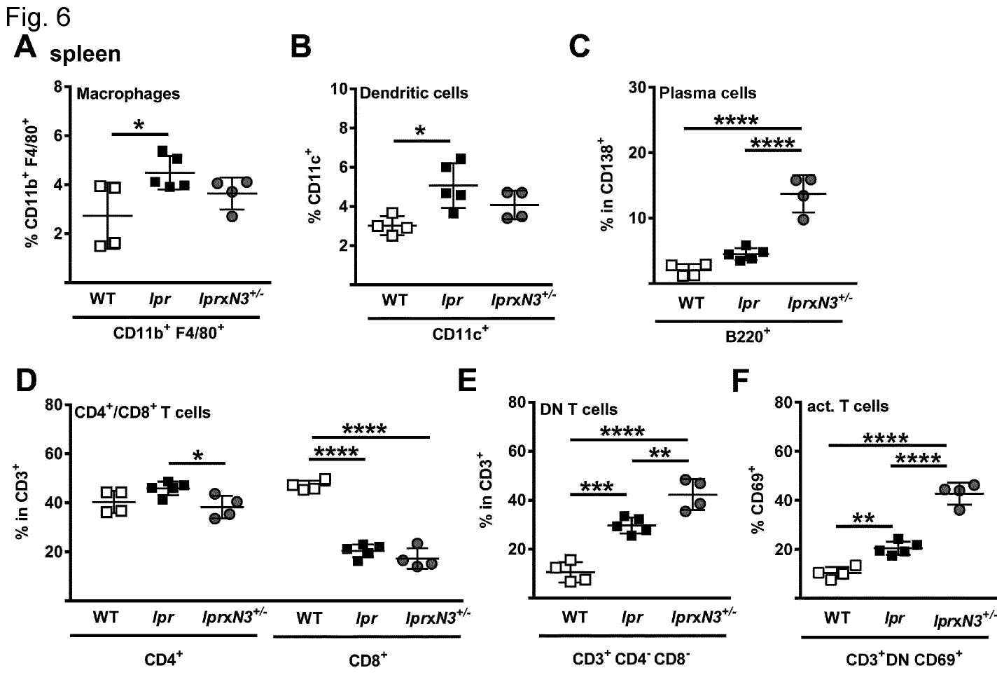

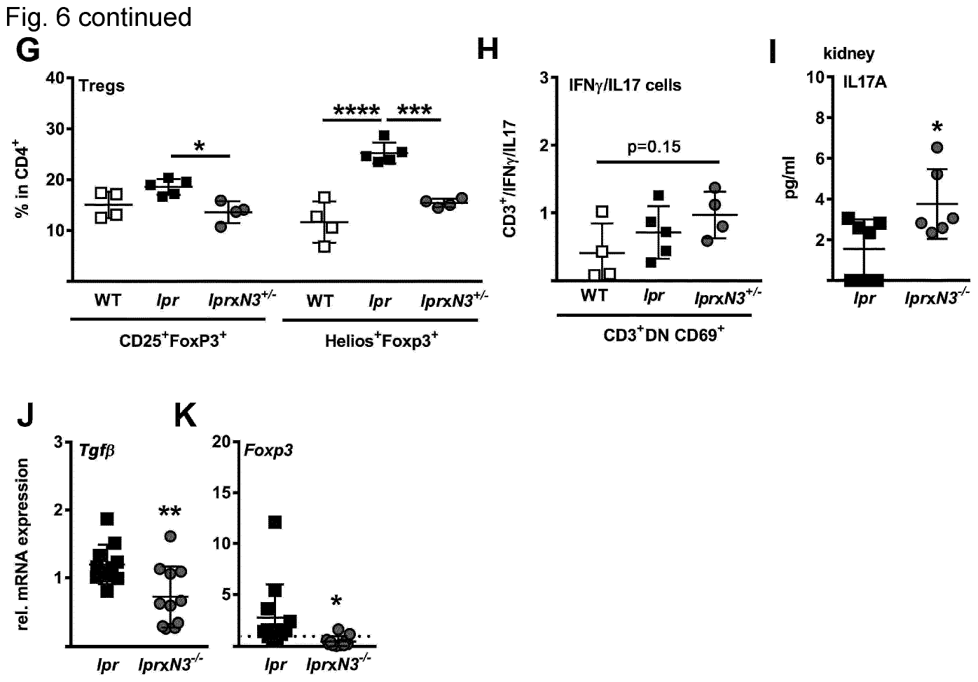

Due to poor breeding yield and the high mortality of B6.lpr×N3^{-/-} mice, it was chosen to analyze immune cell subtypes in younger (18- to 22-weeks old) mice with a heterozygous Notch3 knockout (B6.lpr×N3^{+/-}). B6.lpr animals revealed various SLE-specific changes in their immune cell pattern including enhanced percentages of macrophages (FIG. 6A) and dendritic cells (FIG. 6B), plasma cells (FIG. 6C) and characteristic shifts in several T lymphocyte subtypes (FIG. 6D-G) as compared to B6 mice. In particular, B6.lpr mice had significantly higher levels of CD4/CD8 double-negative T lymphocytes (FIG. 6E), activated CD69⁺ T cells (FIG. 6F) and more regulatory T cells (T_{reg}; FIG. 6G). Double-negative T cells have been described to infiltrate the kidneys and to be the major source of IL-17 production in SLE patients. Indeed, in B6. lpr×N3^{+/-} mice, frequency of IL-17+ cells was numerically higher and renal IL-17 protein content was significantly elevated in B6.lpr×N3^{-/-} as compared to B6.lpr animals (FIGS. 6H/I). Numbers of macrophages and dendritic cells were not significantly changed in B6.lpr×N3^{+/-} mice as compared to B6.lpr mice, whereas percentages of B220+ plasma cells were significantly enhanced. Splenic mRNA levels of both, Tgf/3, a potent inducer of T_{reg} differentiation (FIG. 6J), and Foxp3, a marker of T_{regs} (FIG. 6K), were lower in B6.lpr×N3^{-/-} mice. In summary, high percentages of CD4/CD8 double-negative and activated T cells (FIGS. 6E and 6F) and decreased percentages of T_{regs} (FIG. 6G) indicated a profound disruption of T cell homeostasis even in heterozygous animals compared to B6.lpr mice with constitutive Notch-3 expression.

### Guanidinylated YB-1 Activates Notch-3-Dependent Il10 Expression

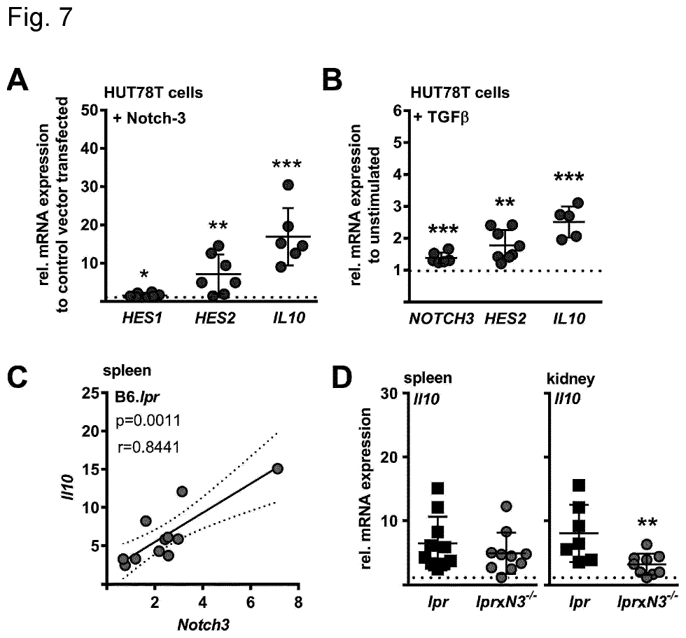

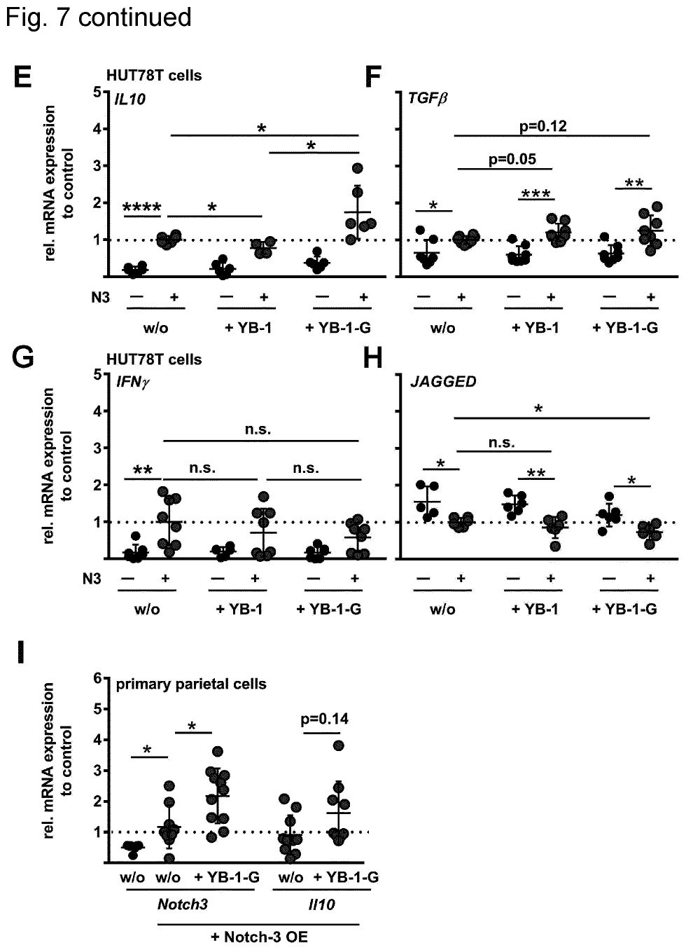

Next, the impact of enforced Notch-3 expression in T lymphocytes was analyzed. Notch-3 overexpression activated its target genes, Hes1 and Hes2, as well as expression of the anti-inflammatory interleukin 10 (Il10) (FIG. 7A). Stimulation with TGF-β, induced Notch3, Hes2 and Il10 expression (FIG. 7B). A strong correlation between Notch3 and Il10 expression was apparent in spleens of B6.lpr mice (FIG. 7C). Genetic deletion of Notch3 resulted in lower Il10 expression in spleens (FIG. 7D, left) and kidneys (FIG. 7D, right) in lupus-prone mice.

Notch-3 overexpression and stimulation with recombinant YB-1 protein harboring the dual-guanidinylation observed in the lupus cohort (YB-1-G-CONTAINING PROTEIN) enhanced expression of anti-inflammatory mediators such as Il10 and Tgfβ in cultivated HUT78T cells (FIGS. 7E/F). In contrast, IFNγ a central pro-inflammatory cytokine of SLE with additional relevance for Th1-specific differentiation, was not further influenced by YB-1-G-CONTAINING PROTEIN stimulation, albeit it was up-regulated upon Notch-3 overexpression (FIG. 7G). Messenger RNA of canonic Notch ligand Jagged1 was down-regulated following Notch-3 overexpression as well as upon exposure to guanidinylated YB-1 (FIG. 7H). In addition, stimulation with YB-1-G-CONTAINING PROTEIN also induced Il10 expression in primary parietal cells isolated from WT mice and forced to overexpress a Notch-3 plasmid, yet without statistical significance (FIG. 7I). Taken together, YB-1-G-CONTAINING PROTEIN mediates anti-inflammatory effects via the Notch-3 receptor.

### DISCUSSION

The conducted study revealed a novel post-translational modification (i.e. guanidinylation) of the immunoregulatory factor YB-1 in lupus-prone mice and SLE patients, and demonstrated that guanidinylated YB-1 acts as a powerful activator of the Notch-3 signaling pathway. Furthermore, Notch-3 activation in SLE exerted protective rather than destructive effects, most likely through modulation of T cell differentiation processes that orchestrate the immune response.

A dysbalance in immune cell compartments is a hallmark of SLE. Although Notch-3 is not strictly necessary for steady-state T lymphopoiesis, it plays a critical role in the differentiation and expansion of T cell subtypes during the immunological response. This is in line with the observation that the immune status of untreated Notch3⁻¹ mice was indistinguishable from their WT littermates. Activated Notch-3, however, affects the T_{reg} response and exerts beneficial effects in murine models of autoimmune diabetes and, as shown here, also in SLE. Mice that express the constitutively active intracellular Notch-3 receptor domains (Notch3-IC Tg mice) show expansion of CD4⁺CD25⁺T_{reg} cells. This is in accordance with the data that genetic depletion of Notch-3 in B6. lpr mice resulted in lower numbers of CD25⁺FoxP3⁺as well as Helios⁺FoxP3⁺ cells. The importance of T_{reg} cells is underscored by the fact that Foxp3-deficient scurfy mice develop lymphoproliferative disorders with largely enhanced Th1, Th2, and Th17 responses. Aberrant lymphocyte proliferation in SLE is also reflected in the fact that particular double negative T cells that are a major source for the production of the pro-inflammatory cytokine IL-17 are expanded in patients with SLE and considerably contribute to the pathogenesis of kidney damage. The present analyses now prove that the double-negative T cell compartment is even further expanded in Notch-3-deficient lupus mice. Not only frequencies, but also the activation status of T cells are further increased in B6.lpr×N3^{-/-}, and the glomerular damage was more pronounced as compared to B6.lpr mice. It appears that individual regulation of the Notch homologues in various organs and cellular compartments differentially affects lupus immunopathogenesis. Whereas a decreased Notch-1 expression in T lymphocytes from SLE patients exerts pro-inflammatory effects and contributes to increased disease activity, increased presence of Notch-3 in the kidneys from lupus nephritis patients counteracts an overwhelming inflammatory state.

LN is the leading cause of morbidity and mortality in patients with SLE. Characteristics of common classes of LN are mesangial inflammation, proliferation of parietal epithelial cells and crescent formation (Lech et al, 2013)¹⁴. Notch-3 is upregulated especially in glomeruli of lupus-prone mice and specimen of human LN patients (FIG. 2). This is consistent with previous observation that Notch3 transcripts were most prominently up-regulated in isolated glomeruli of patients with LN compared with the other protein family members (Notch-1, -2 and -4)². The observation of the soluble, guanidinylated ligand YB-1 (YB-1-G-CONTAINING PROTEIN) in the sera of SLE patients and lupus mice deserves particular attention. Post-translational modifications of YB-1 potently affect its functions as they determine subcellular as well as intra- and extracellular localizations^{11, 15-17}. For the first time, the inventors were able to identify guanidinylation of YB-1 by mass-spectrometry in SLE patients. The degree of YB-1 guanidinylation correlated with SLE disease activity and active kidney involvement. Moreover, YB-1-G-CONTAINING PROTEIN induced Notch-3 expression in primary parietal cells of lupus mice and the Notch-3-mediated gene expression of the anti-inflammatory mediators IL-10 and TGF-β. As such, like Notch-3 itself, YB-1-G-CONTAINING PROTEIN might be considered a protective factor in SLE. Thus, the inventors propose a regulatory loop that points to secreted YB-1-G-CONTAINING PROTEIN as a potent inducer of anti-inflammatory responses, which might be suitable as a future therapeutic strategy. Currently, the underlying mechanisms of YB-1 guanidinylation have not been investigated. A key enzyme in the transamidation of lysine to homoarginine is the arginine:glycine amidinotransferase (AGAT) (Ryan, 1964)¹⁸, which might also convey YB-1 guanidinylation.

Interestingly, the absence of Notch-3 in lupus mice had little effect on the expression of the canonical ligand Jagged. In contrast, overexpression of Notch-3 and stimulation through YB-1-G-CONTAINING PROTEIN even resulted in decreased Jagged expression in T cells. This suggests that soluble ligands play a more prominent role in Notch-3 receptor activation in SLE than canonical ligands. Overexpression of Notch-3 enhances IL-10 production in CD4⁺ T cells and treatment of CD4⁺ T cells with Notch3 antisense DNA results in substantial inhibition of Th1 development. Thus, Notch regulates IL-10 production by Th1 cells under inflammatory conditions and thereby facilitates the self-limitation of Th1 immune responses. As reported earlier, Notch-3 mediates the expansion and function of T_{reg} cells including an increased production of IL-10. This explains very well why the deletion of Notch-3 in SLE may have aggravated disease severity. Notch3^{-/-} mice have been shown to be protected in various models of renal damage such as ischemia/reperfusion (Kavvadas wt al., 2018)¹⁹ and unilateral ureter obstruction (Djudjaj, 2012)². By contrast, in the present study Notch-3 exhibited a renoprotective effect as the deletion of Notch-3 in B6.lpr mice resulted in significantly increased disease severity and significantly increased mortality. In the postnatal period, Notch-3 receptor is required for the elaboration and maintenance of small arterial vessels. The previously observed pro-inflammatory role of Notch-3 could therefore be due to the fact that these acute kidney injuries do not affect the renal vasculature to the same extent. However, vascular lesions in SLE are one of the typical symptoms.

In summary, the protective effect of Notch-3 in SLE might be explained by its role in orchestrating the immune response involving T lymphocyte differentiation, by the induction of anti-inflammatory factors and by receptor activation accomplished via its modified soluble ligand YB-1.

### Material and Methods

### Animal Experiments

C57BL/6 mice were purchased from Charles River, B6.Notch3^{-/-} and MRL. lpr mice from The Jackson Laboratory (Ben Bar Harbor, Me., USA). Animals were held in cages in a room with constant temperature at a 12 hours day/night cycle with access to drinking water and food ad libitum. Animal experiments were approved by the local government authority's review board in accordance with the guidelines for scientific animal experimentation. Female MRL. lpr mice were sacrificed at 8, 12, and 20 weeks of age. Female C57BL/6 (B6), B6.lpr (lpr), B6.Notch3^{-/-} (N3⁻¹), B6.lpr.Notch3^{+/-} (B6.lprxN3^{+/-}; lprxN3^{+/-}) and B6.lpr.Notch3^{-/-} (B6.lprxN3⁻¹; lprxN3⁻¹) mice were sacrificed at the age of 18-22 weeks and 40 weeks, respectively. Their organs as well as urine and blood samples were analyzed using qRT-PCR, immunohistochemistry, immunofluorescence staining, flow cytometry and Western blotting.

### Immunohistochemistry

Samples from kidneys, spleens and lymph nodes were fixed in methyl Carnoy's solution and embedded in paraffin. One μm-thick sections were used for staining of PAS, HE, CD45 (BD Biosciences Heidelberg, Germany) and collagen IV (Southern Biotech, Birmingham, Ala., USA). Biotin-conjugated anti-rabbit/-rat/-goat IgGs (Vector Laboratories, Burlingame, Conn., USA) were used as secondary antibodies and sections were developed with diaminobenzidine substrate with the Vectastain ABC kit (Vector Laboratories). Samples were counterstained with methyl green. For CD45 and collagen IV stainings, the positive areas from 20 images at 200× magnification were subjected to densitometry using ImageJ to calculate a mean area percentage. HE stained spleens and lymph nodes were scanned (Hamamatsu, Herrsching am Ammersee, Germany), and the area of the cross section was analyzed by Aperio ImageScope software (Leica Biosystems, Nussloch, Germany). Proliferation in glomeruli was evaluated semiquantitatively using a score system ranging from 0 to 3 as described before.

### Immunofluorescence

Kidney samples were fixed in 4% formalin or methyl Carnoy's solution and embedded in paraffin. Immunofluorescence staining for CD3 (Bio-Rad, Puchheim, Germany), Notch-3, PDGFR-β (Abcam, Cambridge, UK), CD31, CD44, CD45 (BD Biosciences, Heidelberg, Germany) NG2 and Nestin (Novus Biologicals, Wiesbaden-Nordenstadt) were performed in 3 μm-thick paraffin sections and DAPI (Roche Diagnostics, Mannheim, Germany) was used as staining for the nuclei. Pictures were taken with a Biorevo BZ9000 fluorescence microscope (Keyence, Neu-Isenburg, Germany). Except control biopsies, human biopsies were taken with indication and obtained from a multicenter renal biopsy bank (German Cancer Research Center (DKFZ)). Notch-3 staining was performed in 11 LN patients, 6 patients with IgA nephropathy (IgAN), 5 LN patients (all with RPN/ISN class III or IV), 5 patients with diabetic nephropathy (DN), 5 patients with FSGS and 4 control individuals (3 with post-transplant protocol biopsies and 1 control biopsy). Glomerular Notch-3 expression was evaluated semiquantitatively in LN patients (79 glomeruli), IgAN patients (35 glomeruli) and controls (22 glomeruli) by a researcher who scored the stained area with the following criteria: strong: more than ⅓ of the Bowman's capsule and/or mesangium were positively stained; medium: less than 1/3 were positively stained and w/o: without staining.

### SDS-PAGE and Western Blot

Cell lysis, SDS gel electrophoresis and subsequent blotting was performed as previously described. Anti-GAPDH (NB300-221, Novus Biologicals) and anti-human YB-1 C-terminal (Y0396, Sigma-Aldrich) antibodies were used for detection of specific proteins.

RNA isolation and cDNA synthesis

RNA from cells was isolated using the my-Budget RNA Mini kit (Bio-Budget Technologies, Krefeld, Germany) following the manufacturer's instructions. Preparation of RNA from tissue and subsequent cDNA synthesis was performed as described before.

### Quantitative Real-Time PCR

qRT-PCR was performed as describes elsewhere with following TaqMan probes: human 18S: Hs99999901_s1, Hes1: Hs00172878_m1 Hes2: Hs00219505_m1, IL10: Hs00961622_m1, Jagged1: Hs01070032_m1, and murine 18S: Hs99999901_s1, Hes2: Mm00456108_g1, Hes5: Mm00439311_g1, IL10: Mm00439616_m1, Jagged1: Mm00496902_m1, Notch1: Mm00435249_m1, Notch3: Mm01345646_m1, YB-1: Hs02742754_g1 as well as primers, when performing qPCR with the Core kit for SYBR Green I (Eurogentec, Seraing, Belgium), for

murine GAPDH:
(SEQ ID NO: 2)
5′-GGCAAATTCAACGGCACAGT-3′;
(SEQ ID NO: 3)
5′-AGATGGTGATGGGCTTCCC-3′;
TGF-β:
(SEQ ID NO: 4)
5′-GGACTCTCCACCTGCAAGACC-3′;
(SEQ ID NO: 5)
5′-GGATGGCTTCGATGCGC-3′;
FoxP3:
(SEQ ID NO: 6)
5′-GGCAAATGGAGTCTGCAAGTG-3′;
(SEQ ID NO: 7)
5′-CAGGAGATGATCTGCTTGGCA-3′;
human GAPDH:
(SEQ ID NO: 8)
5′-AGCCACATCGCTCAGACACC-3′;
(SEQ ID NO: 9)
5′-GCGCCCAATACGACCAAA-3′;
TGF-β:
(SEQ ID NO: 10)
5′-ACTACTACGCCAAGGAGGTCAC-3′,
(SEQ ID NO: 11)
5′-TGCTTGAACTTGTCATAGATTTCG-3′,

and a primer assay for IFNγ (Hs_IFNG_1_SG, Qiagen, Hilden, Germany). Measurements were performed in duplicates with the 7300 real-time PCR system (Life technologies, Darmstadt, Germany).

### Flow Cytometry

Single cell suspensions from spleens were obtained using 40 μm cell strainers. After lysis of red blood cells (lysis buffer from eBioscience, Frankfurt, Germany), surface staining was performed with fluorophore-labeled anti-B220, anti-CD3, anti-CD4, anti-CD8, anti-CD11b, anti-CD11c, anti-CD25, anti-CD69, anti-IL17 and anti-F4/80 antibodies. To analyze Foxp3 and Helios expression cells were subsequently fixed, permeabilized and labeled intracellularly with fluorophore-labeled, anti-Helios and anti-FoxP3 antibodies (all from eBioscience) using a FoxP3/transcription factor staining buffer kit (eBioscience) according to the manufacturer's instructions. Flow cytometry was carried out using FACSCanto II device (BD Biosciences, Germany). Data analysis was performed using FCS Express Software.

### Cell Culture

Human T lymphocytes (HUT78T) were cultured and transfected by electroporation with an expression plasmid for the full-length murine Notch-3 receptor as described before. After 24 h, cells were treated with either 10 ng/ml recombinant YB-1 (Abnova, Heidelberg, Germany), the guanidinylated form (YB-1-G-CONTAINING PROTEIN (process described in Rueth et al., 2015 (²⁰), 5 ng/ml TGF-β (ImmunoTools, Friesoythe, Germany) solved in 5 mM hydrochloric acid with 1% BSA, or with solvent alone for an additional 24 h. Isolation, cultivation and transfection of parietal epithelial cells (PECs) from mouse kidneys were performed as follows: For the perfusion, 0.45 g iron(II) oxide was suspended in 50 ml NaCl solution. The mice were sacrificed and the heart was perfused with 20-50 ml iron(II) oxide suspension until the kidneys turned slightly black. Kidneys were removed, and squashed through a 70 μm cell strainer using the plunger of a syringe. The strainer was washed with PBS, the homogenate was collected and sedimented on ice. The sediment was resuspended in 1 ml PBS-Tween (0.05%). The glomeruli were cleaned with a magnetic Eppendorf tube holder. The sediment was washed 4× with PBS-Tween and the glomeruli were suspended in 1 ml of medium (Clonetics EBM with additives, Lonza, Basel, Switzerland) and transferred into a cell culture flask filled with 4 ml of medium. The medium was changed after 10 days without moving the flask. The cells were seeded into 6-well plates and grown until confluency. Subsequently, they were transfected with the Notch-3 overexpressing plasmid using Lipofectamine 2000 (Life Technologies, Carlsbad, Calif., USA) according to the manufacturer's instructions. Briefly, 2.5 μl Lipofectamine and 1.3 μg plasmid are incubated for 5 min in 260 μl FCS-free medium, which was then pipetted to the wells filled with 0.75 ml medium.

### Mass Spectrometry of Serum Proteins and Mass Spectrometry Imaging (MALDI MSI) of Kidney Sections

Human serum samples were collected in accordance to the local ethics regulations and in accordance with the principles of the Declaration of Helsinki. A total of 23 measurements were taken of 17 SLE patients at different times (different states of activity). Serum was prepared as described before (Kork, 2018)²¹ and a MALDI-time of flight (TOF)/TOF MS (Ultraflex III; Bruker Daltonic, Bremen, Germany) MS was performed. A database search (Swiss-Prot) using the Mascot 2.2 search engine (Matrix Science Inc, Boston, Mass.) and Bruker Bio-Tool 3.2 software was performed with the calibrated and annotated spectra to calculate the peptide mass signal for each entry into the sequence database, compare the experimental MALDI-MS and MALDI-MS/MS dataset, and to assign a statistical weight to each individual peptide match using empirically determined factors. Histological sections of 5 μm thickness were made of the kidneys on specially coated slides (indium-tin-oxide coated slides, Bruker Daltonic). The kidney sections were dewaxed, tryptically digested and coated with Matrix (α-cyano-4-hydroxycinnamic acid, Bruker Daltonic) for the MALDI TOF/TOF measurement. The coating with trypsin and matrix were made with a sprayer for MALDI Imaging (HTX TM-Sprayer: TMSP-M3, HTX Technologies, Chapel Hill, USA). The sections were measured with Rapiflex (Bruker Daltonics) and a grid size of 50 μm and the imaging analyses were made with flexlmaging 5.0 (Bruker Daltonics). Also the MS/MS spectra were created with the Rapiflex, followed by a database search (SwissProt) using the Mascot 2.2 search engine (Matrix Science Inc, Boston, Mass.) and Bruker Bio-Tool 3.2 software.

### REFERENCES OF THE EXPERIMENTAL SECTION

1. LaFoya B, Munroe J A, Mia M M, et al. Notch: A multi-functional integrating system of microenvironmental signals. Dev Biol. 2016, 418(2):227-241.
2. Djudjaj S, Chatziantoniou C, Raffetseder U, et al. Notch-3 receptor activation drives inflammation and fibrosis following tubulointerstitial kidney injury. J Pathol. 2012, 228(3):286-299.
3. Mertens P R, Raffetseder U, Rauen T. Notch receptors: a new target in glomerular diseases. Nephrol Dial Transplant. 2008, 23(9):2743-2745.
4. Rauen T, Raffetseder U, Frye B C, et al. YB-1 acts as a ligand for Notch-3 receptors and modulates receptor activation. J Biol Chem. 2009, 284(39):26928-26940.
5. Cervera R, Khamashta M A, Font J, et al. Morbidity and mortality in systemic lupus erythematosus during a 10-year period: a comparison of early and late manifestations in a cohort of 1,000 patients. Medicine (Baltimore). 2003, 82(5):299-308.
6. Davidson A. What is damaging the kidney in lupus nephritis? Nat Rev Rheumatol. 2016, 12(3):143-153.
7. Rauen T, Grammatikos A P, Hedrich C M, et al. cAMP-responsive element modulator alpha (CREMalpha) contributes to decreased Notch-1 expression in T cells from patients with active systemic lupus erythematosus (SLE). J Biol Chem. 2012, 287(51):42525-42532.
8. Alidousty C, Rauen T, Hanssen L, et al. Calcineurin-mediated YB-1 Dephosphorylation Regulates CCLS Expression during Monocyte Differentiation. J Biol Chem. 2014, 289(31):21401-21412.
9. Hanssen L, Alidousty C, Djudjaj S, et al. YB-1 is an early and central mediator of bacterial and sterile inflammation in vivo. J Immunol. 2013, 191(5):2604-2613.
10. Raffetseder U, Liehn E A, Weber C, Mertens P R. Role of cold shock Y-box protein-1 in inflammation, atherosclerosis and organ transplant rejection. Eur J Cell Biol. 2012, 91 (6-7):567-575.
11. Wang J, Djudjaj S, Gibbert L, et al. YB-1 orchestrates onset and resolution of renal inflammation via IL10 gene regulation. J Cell Mol Med. 2017.
12. Gera S, Dighe R R. The soluble ligand Ybox-1 activates Notch3 receptor by binding to epidermal growth factor like repeats 20-23. Arch Biochem Biophys. 2018.
13. Raffetseder U, Rauen T, Boor P, et al. Extracellular YB-1 blockade in experimental nephritis upregulates Notch-3 receptor expression and signaling. Nephron Exp Nephrol. 2011, 118(4):e100-108.
14. Lech M, Anders H J. The pathogenesis of lupus nephritis. J Am Soc Nephrol. 2013, 24(9):1357-1366.
15. Dong W, Wang H, Shahzad K, et al. Activated Protein C Ameliorates Renal Ischemia-Reperfusion Injury by Restricting Y-Box Binding Protein-1 Ubiquitination. J Am Soc Nephrol. 2015.
16. Frye B C, Halfter S, Djudjaj S, et al. Y-box protein-1 is actively secreted through a non-classical pathway and acts as an extracellular mitogen. EMBO Rep. 2009, 10(7):783-789.
17. Wang J, Gibbert L, Djudjaj S, et al. Therapeutic nuclear shuttling of YB-1 reduces renal damage and fibrosis. Kidney Int. 2016, 90(6):1226-1237.
18. Ryan W L, Wells I C. Homocitrulline and Homoarginine Synthesis from Lysine. Science. 1964, 144(3622):1122-1127.
19. Kavvadas P, Keuylian Z, Prakoura N, et al. Notch3 orchestrates epithelial and inflammatory responses to promote acute kidney injury. Kidney Int. 2018, 94(1):126-138.
20. Rueth M, Lemke H D, Preisinger C, et al. Guanidinylations of albumin decreased binding capacity of hydrophobic metabolites. Acta Physiol (Oxf). 2015.
21. Kork F, Jankowski J, Goswami A, et al. Golgin A4 in CSF and granulovacuolar degenerations of patients with Alzheimer disease. Neurology. 2018, 91(19):e1799-e1808.

1. A method for diagnosing lupus nephritis in an individual suffering from systemic lupus erythematosus (SLE), said method conducted in vitro comprising identifying a protein that comprises an YB-1-like cold-shock domain of a sequence homology of at least 96% of SEQ ID NO: 14 which bears one or more guanidinylated lysinyl moieties (YB-1-G) in a sample S obtained from the individual.

2. The method of claim 1, wherein the guanidinylated YB-1-like cold-shock domain (YB-1-G) forms part of an YB-1 protein.

3. The method of claim 1, wherein YB-1-G bears two guanidinylated lysinyl moieties, in particular at positions Lys3 and Lys8.

4. The method of claim 1, wherein the individual is a human and YB-1-G is comprised in human YB-1 protein of SEQ ID NO: 1 which is guanidinylated in amino acid moiety positions Lys53 and Lys58.

5. The method of claim 1, wherein the sample S is:
(a) an extracellular body fluid, in particular wherein the extracellular body fluid is blood serum or a fraction thereof;
(b) a tissue sample; or
(c) a combination of (a) and (b).

6. The method of claim 1, comprising the following steps:
(i) providing the sample S obtained from the individual; and
(ii) determining YB-1-G in the sample S, wherein:
(iia) the presence of YB-1-G in the sample S indicates the presence of lupus nephritis in the individual, or
(iib) an increased level of YB-1-G in the sample S indicates the presence of lupus nephritis in the individual, wherein the level of YB-1-G determined in the sample S is optionally compared with
(a) a predetermined reference value indicating the borderline between a sample S+ indicating the presence of a lupus nephritis and a sample S− indicating the absence of lupus nephritis; and/or
(b) an YB-1-G level determined in a control sample S(0) obtained from a control individual of the same species not having lupus nephritis,
wherein an YB-1-G level determined in the sample S obtained from the individual that is higher than the YB-1-G level borderline between sample S− and S+ and/or at least 50% higher than sample S(0) indicates the presence of lupus nephritis in the individual,
wherein the YB-1-G level in each case is related to the total polypeptide content comprised in the respective sample.

7. The method of claim 1, wherein identifying YB-1-G is detecting YB-1-G by means of detecting the selective binding of an antibody or a fragment thereof to YB-1-G, in particular wherein detecting YB-1-G includes one or both of selected from the group consisting of:
(a) direct immunodetection comprising providing at least one labeled antibody or antibody fragment specific for YB-1-G, and
enabling binding of said labeled antibody or antibody fragment to YB-1-G; and/or
(b) indirect immunodetection comprising providing at least one antibody or antibody fragment specific for YB-1-G and at least one labeled antibody or antibody fragment specifically binding to said antibody or antibody fragment specific for YB-1-G, and
enabling the binding of the antibody or antibody fragment to YB-1-G and the binding of the labeled antibody or antibody fragment to said antibody or antibody fragment specific for YB-1-G.

8. The method of claim 1, wherein identifying YB-1-G includes detecting an autoantibody specific for YB-1-G.

9. The method of claim 1, wherein identifying YB-1-G is conducted by means selected from the group consisting of enzyme-linked immunosorbent assay (ELISA), immuno-electrophoresis, immuno-blotting, Western blot, SDS-PAGE, a lateral flow dipstick, affinity chromatography, flow cytometry, fluorescence-activated cell sorting (FACS), microscopy, and combinations of two or more thereof.

10. The method of claim 1, wherein identifying YB-1-G is conducted by detecting YB-1-G by means of mass spectrophotometry, optionally including fractionation of the YB-1-G in a quadrupole and/or ion trap, wherein mass spectrophotometry is optionally combined with liquid chromatography.

11. An anti-inflammatory and/or immune suppressing compound for use in a method for treating or preventing lupus nephritis in an individual, wherein lupus nephritis has been diagnosed in the individual by means of a method of claim 1.

12. The anti-inflammatory or immune suppressing compound for use of claim 11, wherein the compound is selected from the group consisting of glucocorticoids such as hydrocortisone, prednisolone or dexamethasone, cyclophosphamide, methotrexate, azathioprine or mycophenolatmofetil, calcineurin inhibitors such as cyclosporine, tacrolimus or pimecrolimus, anti-inflammatory drugs (NSAIDs) such as cyclooxygenase (COX) inhibitors, or immunosupressant antibodies.

13. Use of a protein that comprises an YB-1-like cold-shock domain of a sequence homology of at least 96% of SEQ ID NO: 14 which bears one or more guanidinylated lysinyl moieties (YB-1-G) as a biomarker for diagnosing lupus nephritis.

14. An ELISA kit for conducting a method of claim 1, comprising:
(A) an antibody or antibody fragment specific for YB-1-G or an epitope that is specifically bound by an anti-YB-1-G-autoantibody;
(B) optionally an antibody or antibody fragment specifically binding to said antibody or antibody fragment specific for YB-1-G or to said anti-YB-1-G-autoantibody;
(C) optionally one or more suitable buffers for the one or more antibodies or antibody fragments or epitopes;
(D) optionally one or more ELISA plates; and
(E) optionally user instructions.

15. A dipstick usable for the method of claim 1, comprising, placed in the direction of flow of the sample S from the individual, on a carrier that is suitable for soaking the sample S, the following:
(0) an edge or segment suitable for soaking the sample S;
(1) optionally a stripe (1) comprising labeled YB-1-G-specific antibodies or antibody fragments or epitopes that is specifically bound by an anti-YB-1-G-autoantibody which are not immobilized and freely movable when the sample S passes through this stripe (1);
(2) a stripe (2) comprising immobilized YB-1-G-specific antibodies or antibody fragments or an epitope that is specifically bound by an anti-YB-1-G-autoantibody; and
(3) optionally a stripe (3) of immobilized antibodies or antibody fragments specifically binding the one or more components of stripe (1).


SEQUENCE LISTING (14 sequences)

SEQ ID NO: 1 - protein, 324 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 2 - DNA, 20 bp, Artificial Sequence
  primer 1 for murine GAPDH
ggcaaattcaacggcacagt

SEQ ID NO: 3 - DNA, 19 bp, Artificial Sequence
  primer 2 for murine GAPDH
agatggtgatgggcttccc

SEQ ID NO: 4 - DNA, 21 bp, Artificial Sequence
  primer 1 for TGF-beta
ggactctccacctgcaagacc

SEQ ID NO: 5 - DNA, 17 bp, Artificial Sequence
  primer 2 for TGF-beta
ggatggcttcgatgcgc

SEQ ID NO: 6 - DNA, 21 bp, Artificial Sequence
  primer 1 for FoxP3
ggcaaatggagtctgcaagtg

SEQ ID NO: 7 - DNA, 21 bp, Artificial Sequence
  primer 2 for FoxP3
caggagatgatctgcttggca

SEQ ID NO: 8 - DNA, 20 bp, Artificial Sequence
  primer 1 for human GAPDH
agccacatcgctcagacacc

SEQ ID NO: 9 - DNA, 18 bp, Artificial Sequence
  primer 2 for human GAPDH
gcgcccaatacgaccaaa

SEQ ID NO: 10 - DNA, 22 bp, Artificial Sequence
  primer 3 for TGF-beta
actactacgccaaggaggtcac

SEQ ID NO: 11 - DNA, 22 bp, Artificial Sequence
  primer 4 for TGF-beta
tgcttgaacttgtcatagattt

SEQ ID NO: 12 - protein, 20 aa, Homo sapiens
GDKKVIATKVLGTVKWFNVR

SEQ ID NO: 13 - protein, 6 aa, Homo sapiens
KVIATK

SEQ ID NO: 14 - protein, 79 aa, Homo sapiens
DKKVIATKVLGTVKWFNVRNGYGFINRNDTKEDVFVHQTAIKKNNPRKYLRSVGDGETVEFDVVEGEKGAEAANVTGPG